(19) United States
(12) Patent Application Publication
(10) Pub. No.: US 2024/0197621 A1
(43) Pub. Date: Jun. 20, 2024

(54) Buffered Lidocaine Injectable Formulations and Methods for Making Same

(71) Applicant: HIKMA PHARMACEUTICALS USA Inc., Berkeley Heights, NJ (US)

(72) Inventors: Kirankumar R. Chaudhari, Amman (JO); Ahmad A. Abu Sharar, Amman (JO); Ragheb M. AbuRmaileh, Chagrin Falls, OH (US)

(21) Appl. No.: 18/543,158

(22) Filed: Dec. 18, 2023

Related U.S. Application Data
(60) Provisional application No. 63/387,992, filed on Dec. 19, 2022.

(51) Int. Cl. A61K 9/00; A61K 31/167; A61K 47/02

(52) U.S. Cl. CPC A61K 9/0019; A61K 31/167; A61K 47/02

(57) ABSTRACT

A stable liquid formulation for parenteral administration that includes lidocaine or a pharmaceutically acceptable salt thereof. The formulation includes a low concentration of a sodium bicarbonate buffer, sodium chloride, and an optional pH adjuster in an aqueous solution that has a pH of about 6.5 to about 7.2. The liquid formulation is a stable formulation that is supplied as a ready-to-use or product in a container or vial with a carbon dioxide rich headspace.

This application claims priority to and any other benefit of U.S. Provisional Patent Application Ser. No. 63/387,992 filed Dec. 19, 2022, the contents of which are incorporated herein in their entirety by reference.

### FIELD

The invention relates to stable buffered liquid formulations containing lidocaine as an active ingredient and, in particular, an injectable liquid formulation containing lidocaine and a buffer that is stable when stored for extended periods in a pharmaceutically acceptable container.

### BACKGROUND

Lidocaine, chemically known as 2-Diethylamino-N-(2,6-dimethylphenyl)acetamide, is used as a local anesthetic agent, often with a small amount of adrenaline (epinephrine) to prolong its effect. The lidocaine free base is not readily soluble in water and preparations having an increased pH to physiological pH suffer from lidocaine precipitation. Thus, the stability of lidocaine is affected by pH, and the pH of lidocaine solutions is known to be adjusted to 3.3 to 6.5 to minimize precipitation and to prolong shelf life. However, lidocaine solutions at pH 3.3 to 6.5 are also known to increase subjective pain at the injection site and delay anesthetic effect.

To address these issues, health agencies have recommended the formulation of lidocaine with sodium bicarbonate. However, aqueous solutions containing lidocaine, and sodium bicarbonate are known to have an unstable pH well above 7 when formed and can suffer from a drift upwards of pH when stored in vials over time, which can result in unwanted lidocaine precipitation.

Lidocaine hydrochloride is currently available without epinephrine in 1%, 1.5% and 2% injectable solutions in plastic ampules or single dose solutions. The solutions contain methylparaben as an antiseptic preservative and the pH is adjusted to a range of 5.0 to 7.0.

There remains a need for a ready-to-use injectable formulations of lidocaine that offer long-term storage stability without the need for preservatives that also prevent precipitation at pH values near 7.0. The present invention provides stable liquid formulations for injection administration of lidocaine or salts thereof that minimize precipitation and exhibit enhanced storage stability.

### SUMMARY

Described herein are stable injectable liquid formulations of lidocaine or salts thereof that include a sodium bicarbonate buffer, sodium chloride, a carrier (e.g., water for injection) and carbon dioxide gas purging as a pH adjuster and/or in the head space of a container housing the formulations. The formulations have about 1 or about 2 weight percent lidocaine or salt thereof. The formulations have an acidic to neutral pH in the range of about 6.5 to about 7.4, and preferably about 6.5 to about 7.3 or less than 7.2. The carbon dioxide purging of the injectable liquid formulation or bulk liquid formulation used to provide the same can be the sole step for adjusting pH without the need for one or more additional pH adjusters in the formulation. As evidenced in the present disclosure, the formulations exhibit stability over long-term and accelerated storage conditions, for example, such that one or more of the following properties are exhibited, the injectable liquid formulation has not more than 600 particulates, not more than 100 particulates, not more than 75 particulates, not more than 50 particulates, not more than 25 particulate, not more than 20 particulates, or not more than 15 particulates having an average diameter of greater than 25 μm after storage for 3 months at 40° C./75 relative humidity (RH), the injectable liquid formulation has not more than 0.2, not more than 0.1 or not more than 0.06 weight percent of total impurities after storage for 3 months at 40° C./75 RH based on the total weight of the formulation, excluding baseline total impurity measurement upon forming the formulation, the injectable liquid formulation retains about 95% or more, about 96% or more, about 98% or more or about 99% or more of the initial concentration of the lidocaine or a pharmaceutically acceptable salt thereof after storage for 3 months at 40° C./75 RH, the injectable liquid formulation retains about 95% or more of the sodium bicarbonate buffer after storage for 3 months at 40° C./75 RH, and the injectable liquid formulation has not more than 600 particulates, not more than 400 particulates, not more than 200 particulates, or not more than 175 particulates having an average diameter of greater than 10 μm after storage for 3 months at 40° C./75 RH.

In a first aspect, there is disclosed an injectable liquid formulation in a container, the formulation includes lidocaine or a pharmaceutically acceptable salt thereof present at a concentration of about 5 to about 10 gm/mL, about 7.5 to about 10 mg/mL or 15 to 20 mg/mL as the sole pharmaceutically active agent; sodium bicarbonate buffer present at a concentration of about 4 to about 17 mg/mL or about 6 to about 12 mg/mL; and a carrier, for example, an aqueous carrier or water for injection present at more than 90% of the weight of the injectable liquid formulation, wherein the injectable liquid formulation has a pH of about 6.5 to about 7.2 and the container comprises a gas head space, the gas head space includes greater than 30, 40 or 50 weight percent carbon dioxide

In an example of aspect 1, the injectable liquid formulation exhibits not more than 600 particulates having an average diameter of greater than 10 μm after storage for 3 months at 40° C. in the container.

In an example of aspect 1, the lidocaine or a pharmaceutically acceptable salt thereof is lidocaine hydrochloride, lidocaine hydrobromide, lidocaine oxalate, lidocaine fumarate, lidocaine adipate, lidocaine maleate, lidocaine malonate or lidocaine tosylate.

In another example of aspect 1, the lidocaine hydrochloride is present at a concentration of about 5 mg/mL, about 9 mg/mL or 18 mg/mL in the injectable liquid formulation.

In another example of aspect 1, the weight ratio of the sodium bicarbonate buffer to the lidocaine or a pharmaceutically acceptable salt thereof is in the range of about 0.3:1 to about 1.4:1, about 0.4:1 to about 1.3:1, about 0.5:1 to about 1.2:1, about 0.6:1 to about 1.1:1, or about 0.7:1 to about 0.9:1, or about 1:1 or 0.8:1.

In another example of aspect 1, the weight ratio of the sodium bicarbonate buffer to the lidocaine hydrochloride is in the range of about 0.8:1 to about 0.9:1 or about 0.84:1.

In another example of aspect 1, the sodium bicarbonate is the sole buffer in the injectable liquid formulation, or the buffer consists of sodium bicarbonate.

In another example of aspect 1, the container has a sealed internal volume, and the sealed internal volume consists of the injectable liquid formulation and the gas head space. The sealed internal volume of the container can be formed by a vial sealed with a stopper.

In another example of aspect 1, the gas head space comprises greater than 70% carbon dioxide.

In another example of aspect 1, the container has a sealed internal volume, and the gas head space is between 10 and 50 volume percent of the sealed internal volume of the container. For example, a vial sealed with a stopper has a sealed internal volume that is occupied by the injectable liquid formulation and the remaining gas head space.

In another example of aspect 1, the formulation further includes sodium chloride present at a concentration of about 1 to about 8 mg/mL.

In another example of aspect 1, the formulation further includes one or more pH adjusters. The one or more pH adjusters can be in addition to adjustment of the pH of the injectable liquid formulation with carbon dioxide gas purging.

In another example of aspect 1, the injectable liquid formulation has a pH of about 6.5 to about 7.2, about 6.7 to about 7, or less than or equal to 7.

In another example of aspect 1, the pH of the injectable liquid formulation in the container remains within about 5% of an initial pH of the injectable liquid formulation when formed as compared to a measured pH of injectable liquid formulation after storage for 3 months at 40° C. in the container.

In another example of aspect 1, the injectable liquid formulation exhibits not more than 6000 particulates having an average diameter of greater than 10 μm after storage for 3 months at 40° C.

In another example of aspect 1, injectable liquid formulation has not more than 0.2 weight percent of total impurities after storage for 3 months at 40° C.

In another example of aspect 1, the injectable liquid formulation retains about 95% or more of the initial concentration of the lidocaine or a pharmaceutically acceptable salt thereof after storage for 3 months at 40° C.

In another example of aspect 1, the injectable liquid formulation retains about 95% or more of the sodium bicarbonate buffer after storage for 3 months at 40° C.

In another example of aspect 1, the injectable liquid formulation is a ready-to-use or ready-to-administer formulation.

In another example of aspect 1, the container is a glass vial.

In another example of aspect 1, the container has a sulfur-treated surface that contacts the injectable liquid formulation. The sulfur-treated surface is the chamber surface of the vial and encompasses the internal volume of the vial.

In another example of aspect 1, the injectable liquid formulation is free of one or more of the components selected from the group consisting of a chelating agent, a saccharide, a calcium salt, a sulfate, a sulfide, a sugar and a sugar alcohol. For example, the injectable liquid formulation includes no chelating agent, saccharide, calcium salt compound, sulfate, sulfide, sugar and sugar alcohol.

In another example of aspect 1, the injectable liquid formulation includes no more than 1000 particulates having an average diameter of greater than 10 μm after three consecutive freeze-thaw cycles or no more than 100 particulates having an average diameter of greater than 25 μm after three consecutive freeze-thaw cycles, wherein each freeze-thaw cycle consists of storing the injectable liquid formulation in a vial for 2 days at −20° C. and at 40° C. at 75% relative humidity for 2 days.

In another example of aspect 1, the injectable liquid formulation is subjected to a pH adjustment step using carbon dioxide prior to being filled in the container. For example, the bulk solution of the same composition as the injectable liquid formulation is subjected to carbon dioxide purging prior to filling the container with a portion of the bulk solution to form the injectable liquid formulation of aspect 1.

In another example of aspect 1, the bulk solution used to fill the container with the injectable liquid formulation, prior to filling the container, is purged with carbon dioxide to adjust the pH of the bulk solution to 7.5 or less, 7.4 or less, 7.3 or less, 7.2 or less, 7.1 or less or 7.0 or less. The initial pH of the injectable liquid formulation filled into the container from the bulk solution is in the range of 6.7 to 7.3.

In another example of aspect 1, the sole method of adjusting the pH of the bulk solution used to fill the container with the injectable liquid formulation constitutes subjecting the bulk solution to carbon dioxide. For instance, the pH of the bulk solution, after its formation, is not adjusted with a pH adjuster (e.g., hydrogen chloride or sodium hydroxide).

In another example of aspect 1, the head space in the container housing the injectable liquid formulation, after being filled with the bulk solution that was subjected to carbon dioxide (e.g., carbon dioxide purging) to adjust its pH, is not purged with carbon dioxide gas prior to sealing the container and its head space. Alternatively, the head space in the container housing the injectable liquid formulation is purged with carbon dioxide to create a head space having a carbon dioxide content of 20 or more, 30 or more, 40 or more, 50 or more, 60 or more, 70 or more or 80 or more volume percent.

In another example of aspect 1, the bulk solution of the same composition as the injectable liquid formulation is not subjected to carbon dioxide purging prior to filling the container with a portion of the bulk solution to form the injectable liquid formulation of aspect 1, but the head space in the container housing the injectable liquid formulation, after being filled with the bulk solution, is purged with carbon dioxide to create a head space having a carbon dioxide content of 20 or more, 30 or more, 40 or more, 50 or more, 60 or more, 70 or more or 80 or more volume percent.

In a second aspect, there is disclosed a ready-to-use injectable liquid formulation in a container, the formulation including lidocaine hydrochloride present at a concentration of about 9 to about 10 mg/mL or 18 to 20 mg/mL, sodium bicarbonate buffer present at a concentration of about 4 to about 17 mg/mL or about 6 to about 12 mg/mL, and water for injection, wherein the container has a gas head space purged with carbon dioxide prior to scaling the container, the gas head space of the sealed container has greater than 70 weight percent carbon dioxide, wherein the injectable liquid formulation has an initial pH of about 6.5 to about 7 when formed and the initial pH remains within about 5% of its value after storage for 3 months at 40° C. in the container, and wherein the injectable liquid formulation exhibits not more than 500 particulates having an average diameter of greater than 10 μm after storage for 3 months at 40° C. in the container.

In an example of aspect 2, the injectable liquid formulation further exhibits after storage for 3 months at 40° C. at least one of the properties of not more than 600 particulates having an average diameter of greater than 25 μm, not more than 0.2 weight percent of total impurities, about 95% or more of the initial concentration of the lidocaine hydrochloride, and about 95% or more of the sodium bicarbonate buffer.

In another example of aspect 2, the injectable liquid formulation is a stable ready-to-use or ready-to-administer formulation.

In another example of aspect 2, the container is a vial having a volume for housing the injectable liquid formulation in that the volume has a sulfur-treated surface in contact with the injectable liquid formulation.

In another example of aspect 2, the container has a sealed internal volume, and the gas head space is between 10 and 60, between 30 and 60, or between 30 and 50 volume percent of the sealed internal volume of the container.

In another example of aspect 2, the lidocaine hydrochloride is present as the sole pharmaceutically active agent in the injectable liquid formulation.

In a third aspect, there is disclosed a method of preparing an injectable liquid formulation, the method includes providing a bulk liquid formulation that contains lidocaine hydrochloride present at a concentration of about 7.5 to about 20 mg/mL as the sole pharmaceutically active agent; sodium bicarbonate buffer present at a concentration of about 4 to about 17 mg/mL or about 6 to about 12 mg/mL; and water for injection present at more than 90% of the weight of the injectable liquid formulation, wherein the bulk liquid formulation has a pH above 7.2; filling a portion of the bulk liquid formulation into a container under a blanket of carbon dioxide; and then sealing the container to provide an airtight enclosure that has a gas head space, the gas head space having greater than 50% carbon dioxide.

In an example of aspect 3, the portion of the bulk liquid formulation filled into the container is 10 mL or 20 mL.

In another example of aspect 3, the bulk liquid formulation has a pH above 7.2 and the method includes the step of purging the bulk liquid formulation with carbon dioxide for a sufficient period of time to reduce the pH of the bulk liquid to below 7.0, for example, 6.5 to 6.8, prior to filling the portion of the bulk liquid formulation into the container.

In another example of aspect 3, the bulk liquid formulation does not contain a pH adjuster when it is used to fill the portion of the bulk liquid formulation into the container and the formulation in the sealed container is free of a pH adjuster such that the sole pH adjustment of the liquid injectable formulation in the sealed container is the step of purging the bulk liquid formulation with carbon dioxide gas.

In another example of aspect 3, the pH of the bulk liquid formulation is in the range of 6.5 to 6.9 after being purged with carbon dioxide gas and before filling a portion of the bulk liquid formulation into the container under a carbon dioxide rich blanket.

In another example of aspect 3, the gas head space of the container has greater than 70% carbon dioxide.

In another example of aspect 3, the container has a sealed internal volume, and the gas head space portion of the sealed internal volume is between 10 and 60 percent of the sealed internal volume of the container.

In another example of aspect 3, the container has a sulfur-treated surface that contacts the injectable liquid stored in the container.

In another example of aspect 3, the container is a glass vial sealed with a stopper, for example, a rubber stopper.

In another example of aspect 3, the injectable liquid formulation in the sealed container has a pH of less than 7.

In another example of aspect 3, the injectable liquid formulation is a ready-to-use or ready-to-administer formulation.

In another example of aspect 3, the injectable liquid formulation in the sealed container has an initial pH of about 6.5 to about 7 after filling and the initial pH remains below 7 after storage of the container for 3 months at 40° C.

In another example of aspect 3, the initial pH is in the range of 6.5 to 6.8 and the initial pH is 6.8 or below after storage of the container for 3 months at 40° C.

In another example of aspect 3, the bulk liquid formulation further comprising a pH adjuster and the sodium bicarbonate is the sole buffer in the bulk liquid formulation.

In a fourth aspect, there is disclosed a ready-to-use injectable liquid formulation in a container, the formulation including lidocaine or an acceptable salt thereof (e.g., lidocaine hydrochloride) present at a concentration of about 10, sodium bicarbonate buffer present at a concentration of about 8 to about 9 mg/mL, water for injection and optionally sodium chloride at a concentration of about 0.5 to about 2 mg/mL, wherein the container has a gas head space purged with carbon dioxide prior to sealing the container, the gas head space of the sealed container has greater than 40 weight percent carbon dioxide, wherein the injectable liquid formulation has an initial pH of about 6.5 to about 7.3 when formed and the initial pH remains within about 5% of its value after storage for 3 months at 40° C./75 RH in the container, and wherein the injectable liquid formulation exhibits not more than 200 particulates having an average diameter of greater than 10 μm or not more than 20 particulates having an average diameter of greater than 25 μm after storage for 3 months at 40° C./75 RH in the container.

In another example of aspect 4, the injectable liquid formulation further exhibits after storage for 3 months at 40° C./75 RH at least one of the properties of not more than 0.05 weight percent of total impurities, about 98% or more of the initial concentration of the lidocaine hydrochloride, and/or about 98% or more of the sodium bicarbonate buffer

Any one of the above aspects (or examples of those aspects) may be provided alone or in combination with any one or more of the examples of that aspect discussed above; e.g., the first aspect may be provided alone or in combination with any one or more of the examples of the first aspect discussed above; and the second aspect may be provided alone or in combination with any one or more of the examples of the second aspect discussed above; and so-forth.

Additional features and advantages will be set forth in the detailed description which follows, and in part will be readily apparent to those skilled in the art from that description or recognized by practicing the embodiments as described herein, including the detailed description which follows, and the claims. It is to be understood that both the foregoing general description and the following detailed description are merely exemplary, and are intended to provide an overview or framework to understanding the nature and character of the claims.

### DETAILED DESCRIPTION

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment.

Herein, when a range such as 5-25 (or 5 to 25) is given, this means preferably at least or more than 5 and, separately and independently, preferably not more or less than 25. In an example, such a range defines independently 5 or more, and separately and independently, 25 or less.

The present disclosure is directed to stable liquid aqueous formulations as a medical and pharmaceutical preparation. The formulations are for injection administration and the formulations include lidocaine or a pharmaceutically acceptable salt thereof, a sodium bicarbonate buffer and a carrier, for instance, an aqueous carrier or water for injection. The formulations can optionally include sodium chloride in the aqueous solution and one or more pH adjusters. The formulations have a pH in the range of about 6.5 to about 7.2. The formulations are stable when stored and pH drift outside of the range 6.5 to 7.2 is controlled.

The formulations of lidocaine are ready-to-use or ready to administer formulations for injection administration and can be used without the need for additional ingredient mixing prior to administration. A ready-to-use or ready-to-administer formulation is a sterile, liquid injectable formulation that may not require mixing before use, for example, with another active ingredient or buffer, such that the formulation can be directly administered or further diluted if present as a concentrated solution. For example, a ready-to-administer formulation can include the required concentration and volume in the final container such as a syringe, vial, ampule or injector. Administration of the formulations may be performed by subcutaneous injection, intramuscular injection, percutaneous injection, intradermal injection or intravenous injection by means of a syringe, optionally a pen-like syringe. Carriers or diluents, if used, can include, for instance, fluids suitable for parenteral administration such as water for injection or sodium chloride solutions.

In one or more embodiments, the formulations of lidocaine are ready-to-use formulations that can be admixed with another solution or active agent prior to administration. For instance, the lidocaine ready-to-use formulations can be mixed with epinephrine if desired to form an admixture of two active ingredients. Epinephrine is a catecholamine, a sympathomimetic monoamine derived from the amino acids phenylalanine and tyrosine, and is a known vasoconstrictor and has beneficial effects when combined with lidocaine.

The formulations can be administered for local or regional anesthesia, for instance, by injection and intravenous administration. Regional anesthesia can include peripheral nerve block uses, for instance, brachial plexus and intercostal, and by central neural uses that can include lumbar and caudal epidural blocks. The formulations of the present disclosure are suitable for injection administration, for example, to a mammal to initiate local or regional anesthesia. Preferably, the mammal is a human.

The lidocaine liquid injectable formulations of the present disclosure are stable or exhibit stability (e.g., active agent assay, impurity profile, precipitation of particulates) when stored. Storage of the formulations in a sealed contain can result in stability concerns, which includes formulation properties that may be affected by storage conditions, for example, active ingredient strength or concentration, impurities (e.g., individual components and total), visual appearance characteristics (e.g., color, clarity, cloudy, haze, precipitates, etc.) and pH drift that can trigger precipitation, concentration loss and impurity formation. Storage conditions that may affect stability can include, for example, storage temperature, humidity (e.g., relative), and storage time period.

In one or more embodiments, stability can be measured by, for example, the amount of total impurities, inclusive of degradation products, that are formed after formation of the lidocaine formulations for a specified period of time at specified storage conditions (e.g., temperature, humidity) in a container minus the initial total impurities as measured following formation as an initial measurement. In one or more embodiments, a liquid injectable lidocaine formulation includes a formulation that retains about 95% or more, about 96% or more, about 97% or more, about 98% or more, about 98.5% or more, or about 99% or more of the initial concentration of lidocaine or pharmaceutically acceptable salt thereof in the formulation after storage (1 month, 2 months, 3 months) under standard (e.g., 25° C.) or accelerated conditions (e.g., 2-8° C., 40° C., 50° C.) at a relative humidity in the range of 60 to 75%. Stability can also be measured by maintaining the desired concentration of sodium bicarbonate buffer present in the formulation. In one or more embodiments, a liquid injectable lidocaine formulation includes a formulation that retains about 95% or more, about 96% or more, about 97% or more, about 98% or more, about 98.5% or more, or about 99% or more of the initial concentration of sodium bicarbonate buffer in the formulation after storage (1 month, 2 months, 3 months) under standard (e.g., 25° C.) or accelerated conditions (e.g., 2-8° C., 40° C., 50° C.) at a relative humidity in the range of 60 to 75%.

Stability of a lidocaine formulation is measured over time after an initial time point measured shortly after formation of the formulation. The initial measurements are taken within 24 hours of the filling of the formulation into a pharmaceutically acceptable container (e.g., a vial) that is sealed and used for storage. In one or more embodiments, a stable lidocaine formulation includes a formulation that contains about 0.15% or less, about 0.2% or less, about 0.25% or less, about 0.3% or less, or about 0.5% or less of an individual impurity (e.g., a degradation impurity or lidocaine-related degradation impurity) formed after formation of the formulation and present after storage under standard or accelerated conditions (e.g., about 25° C., about 2-8° C. and about 40° or 50° C., all at a relative humidity in the range of 60 to 75%) for about 1, about 2, about 3, or about 3 or more months. Any measured individual impurity for purposes of measuring stability of a formulation herein does not include any impurity present in any ingredient prior to formation of the lidocaine formulation. That is, as used herein, an impurity or impurities in an invented formulation refers to any impurity, including a degradation product, formed after formation of the formulations, for example, upon storage of the formed formulation in a pharmaceutically acceptable container such as an airtight sealed glass vial.

In one or more embodiments, the formulations may contain ropivacaine as an impurity at not more than a measured concentration at a predetermined temperature and time period. For example, the formulation can contain not more than 0.1% ropivacaine, not more than about 0.05% ropivacaine, or not more than about 0.01% ropivacaine, as measured after storage of the formulation at about 2-8° C., 25° C., 40° C., or 50° C. for a period of 1, 2 or 3 months. Relative humidity of the storage conditions can include 60% and up to 75%.

In one or more embodiments, a stable lidocaine formulation includes a formulation that contains about 0.15% or less, about 0.2% or less, about 0.25% or less, about 0.3% or less, about 0.5% or less, or about 1% or less of total impurities or lidocaine degradation product present after storage under standard or accelerated conditions. Impurities and degradation products, whether individual or total, can be measured by conventional methods, for example, liquid chromatography (HPLC). In an example, a stable lidocaine formulation includes about 0.5% or less, about 0.3% or less, about 0.2% or less, or about 0.15% or less of total impurities after storage of the formulation at about 25° C. for a period of 3 months. In another example, a stable lidocaine formulation includes about 0.5% or less, about 0.3% or less, about 0.2% or less, or about 0.15% or less of total impurities after storage of the formulation at about 40° C. for a period of 3 months.

In one or more embodiments, a liquid injectable lidocaine formulation includes a formulation that is stable for about 3 months or more, about 6 months or more when stored at an accelerated temperature of about 25° or 40° C. In one or more embodiments, a liquid injectable lidocaine formulation includes a formulation that is stable for about 6 months or more, or about 12 months or more when stored at about room temperature (i.e. 25° C.).

In other embodiments, a liquid injectable lidocaine formulation includes a particulate count of particles below industry standards. For example, the liquid injectable formulations of the invention can have less than 6,000, less than 5,000, less than 4,000, less than 3,000, less than 2,000, less than 1,000, less than 800, less than 500, less than 400, less than 300 or less than 200 sub-visible particles per stored formulation such as a ready-to-used formulation in a container, with the particles having a size greater than 10 μm after storage for 1, 3 or 6 months at 25° or 40° C. and at a relative humidity in the range of 60 to 75% in a container. The number of particles can be determined according to USP 27.

<788> Particulate matter in injections by light obscuration particle count test. In another example, the liquid injectable formulations can have less than 600, less than 500, less than 400, less than 300, less than 200, less than 100, less than 70, less than 50, less than 40 or less than 30 particles having a size greater than 25 μm after storage for 1 or 3 months at 25° or 40° C. and at a relative humidity in the range of 60 to 75% in a container. The measured particles can be defined as having an average diameter. In other embodiments, the number of particles having a size greater than 10 μm increases from an initial concentration of particles when the formulation is formed to a concentration after storage of the formulation for 3 months at 25° and 40° C. in a container, the increase is, respectively, less than 70%, less than 50%, or less than 40% of the initial concentration of particles and less than 125%, less than 100%, or less than 90% of the initial concentration of particles. In yet other embodiments, the number of particles having a size greater than 25 μm increases from an initial concentration of particles when the formulation is formed to a concentration after storage of the formulation for 3 months at 25° and 40° C. in a container, the increase is, respectively, less than 70%, less than 60%, or less than 50% of the initial concentration of particles and less than 50%, less than 40%, or less than 30% of the initial concentration of particles.

In other embodiments, the injectable liquid formulations exhibit stability as measured by having particulate counts below industry standards. For example, the liquid injectable formulations of the invention can have less than 6,000, less than 5,000, less than 4,000, less than 3,000, less than 2,000, less than 1,000, less than 800, less than 500, less than 400, less than 300 or less than 200 sub-visible particles per stored formulation, such as a ready-to-used formulation in a container, with the particles having a size greater than 10 μm after at least three consecutive freeze-thaw cycles such that each freeze-thaw cycle consists of storing the injectable liquid formulation for 2 days at −20° C. and then at 40° C. at 75% relative humidity for 2 days. The number of particles can be determined according to USP 43, <788> Particulate matter in injections by light obscuration particle count test. In another example, the liquid injectable formulations can have less than 600, less than 500, less than 400, less than 300, less than 200, less than 100, less than 70, less than 50, less than 40 or less than 30 particles having a size greater than 25 μm after at least three consecutive freeze-thaw cycles such that each freeze-thaw cycle consists of storing the injectable liquid formulation for 2 days at −20° C. and at 40° C. at 75% relative humidity for 2 days. The measured particles can be defined as having an average diameter.

The formulations of the present disclosure contain, as the active ingredient, lidocaine or any pharmaceutically acceptable salt thereof. In some embodiments, the formulations preferably contain lidocaine or any pharmaceutically acceptable salt thereof as the sole active ingredient characterized in that no other active ingredients, for instance epinephrine, heparin, or acetylcysteine, are present or detectable in the formulation. In one example, the formulation contains lidocaine hydrochloride or HCl. Other suitable salts of lidocaine include, for example, lidocaine hydrobromide, lidocaine oxalate, lidocaine fumarate, lidocaine adipate, lidocaine maleate, lidocaine malonate or lidocaine tosylate. The lidocaine or salt thereof, for instance lidocaine HCl, can be present in the formulation at a concentration of about 7 mg/mL (milligrams/milliliter) or more, about 8 mg/mL or more, about 9 mg/mL or more, or about 11 mg/mL or less or about 10 mg/mL or less. In some embodiments, the liquid injectable formulations contain a concentration of about 5 mg/mL, about 7.5 mg/mL, about 8 mg/mL, about 8.5 mg/mL, about 9 mg/mL, about 9.1 mg/mL or about 9.5 mg/mL of lidocaine or a pharmaceutically acceptable salt thereof. In one or more embodiments, the liquid lidocaine formulations contain about 5 mg, about 6 mg, about 7 mg, about 7.5 mg, about 7.9 mg about 8 mg, about 9 mg or about 10 mg of lidocaine or a pharmaceutically acceptable salt thereof per storage container (e.g., vial).

In other embodiments, the lidocaine or salt thereof, for instance lidocaine HCl, can be present in the formulation at a concentration of about 14 mg/mL (milligrams/milliliter) or more, about 16 mg/mL or more, about 18 mg/mL or more, or about 22 mg/mL or less or about 20 mg/mL or less. In some embodiments, the liquid injectable formulations contain a concentration of about 15 mg/mL, about 16 mg/mL, about 17 mg/mL, about 18 mg/mL, about 18.2 mg/mL or about 19 mg/mL of lidocaine or a pharmaceutically acceptable salt thereof. In one or more embodiments, the liquid lidocaine formulations contain about 10 mg, about 12 mg, about 14 mg, about 15 mg, about 15.8 mg, about 16 mg, about 18 mg or about 20 mg of lidocaine or a pharmaceutically acceptable salt thereof per storage container (e.g., vial).

In one or more embodiments, the formulation volume (e.g., amount of liquid in a storage container) is about 1 mL or more, about 2 mL or more, about 3 mL or more, about 4 mL or more, about 5 mL or more, about 6 mL or more, about 7 mL or more, or about 8 mL or more. In one or more embodiments, the formulation volume is about 25 mL or less, about 22 mL or less, about 20 mL or less, about 18 mL or less, about 15 mL or less, or about 11 mL or less. For example, the formulation volume can be about 5 mL to about 15 mL, about 8 mL to about 12 mL, about 9 mL to about 11 mL, about 10 mL. In another example, the formulation volume can be about 30 mL to about 25 mL, about 22 mL to about 21 mL, about 20 mL to about 19 mL, about 18 mL. Appropriate-sized containers for storing formulation volumes can be determined by one of ordinary skill in the art. In one or more embodiments, the formulation volume can occupy 40% or more, 45% or more, 50% or more, 60% or more, 70% or more, 80% or more or 90% or more of the total volume of the container. In other embodiments, the formulation volume can occupy 90% or less, 80% or less, 70% or less, 60% or less, 55% or less or 50% or less of the total volume of the container. In an example, the formulation volume can be 5 mL and stored in a 10 mL vial or be 10 mL and stored in a 20 mL vial such that the formulation volume is 50% of the total volume of the container with the remaining container volume being head space (5 mL, 10 mL) that can have a select carbon dioxide content as described herein.

The formulations for injectable administration can be stored in or supplied in any suitable container. For example, the formulation can be in a container that includes, but is not limited to, a vial (e.g., single or multidose vials), ampoule, bottle, or syringe (e.g., pre-filled syringe or component of an auto-injector). The container can be made of any suitable material, for instance, glass, plastic, or rubber, although glass is preferred. The container (e.g., vial) can have any suitable volume for storing the formulation, for example, the container can have a volume of 5 mL, 10 mL, 15 mL, 20 mL, 25 mL, 30 mL, 35 mL or 40 mL.

Prior to filling the formulation in a container, the container can be sterile and has been subjected to a sterilization process prior to filing with the sterile formulations of the invention. Containers are sealed as typical in the industry, for example, with the use of a lid, cap, closure, stopper and the like. The containers also can be coated or treated with one or more components to reduce degradation and reaction with ingredients of the formulation and prevent pH drift. For example, a glass container can have a sulfur-treated surface for contacting the formulation to reduce or prevent change in pH of the formulation during storage. In another example, a container surface in contact with the formulation can be coated with silicon or a vial with a treated inner surface for storing the formulation can be used. Optionally, to shield the formulation from exposure to light, a container can optionally be opaque or tinted with a color, and preferably stored in a box for transport or shelving. For instance, amber- or flint-colored vials are suitable containers.

The formulations further include sodium chloride (NaCl) as an inorganic salt. Solutions of 0.9% sodium chloride are known as physiological saline. Saline can aid in maintaining osmotic pressure, function as an acid-base balance at localized pain sites, and also prevent dehydration. The sodium chloride can be present in the liquid injectable formulation at a concentration of about 0.5 mg/mL (milligrams/milliliter) or more, about 0.75 mg/mL or more, about 1 mg/mL or more, about 1.5 mg/mL or more, about 2 mg/mL or more, about 3 mg/mL or more, about 4 mg/mL or more, about 5 mg/mL or more, about 6 mg/mL or more or about 12 mg/mL or less, about 10 mg/mL or less, about 8 mg/mL or less, about 7 mg/mL or less, about 6 mg/mL or less, about 5 mg/mL or less, about 4 mg/mL or less or about 3 mg/mL or less. In some embodiments, the liquid injectable formulations contain a sodium chloride concentration of about 6 mg/mL, about 6.1 mg/mL, about 6.2 mg/mL, about 6.3 mg/mL, about 6.4 mg/mL or about 6.5 mg/mL. In some embodiments, the liquid injectable formulations contain a sodium chloride concentration of about 0.8 mg/mL, about 0.9 mg/mL, about 1.0 mg/mL, about 1.1 mg/mL or about 1.2 mg/mL. In one or more embodiments, the liquid lidocaine formulations contain about 4 mg to 100 mg, about 5 mg to about 80 mg, about 6 mg to about 70 mg, about 6.2 mg to about 68 mg, about 6.3 mg to about 65 mg, or about 6.5 mg to about 63 or 64 mg of sodium chloride per storage container (e.g., vial). In one or more embodiments, the liquid lidocaine formulations contain about 0.1 mg to 10 mg, about 0.5 mg to about 5 mg, about 0.6 mg to about 4 mg, about 0.7 mg to about 4 mg, about 0.8 mg to about 2 mg, about 0.9 mg to about 1.5 mg or about 1.0 mg to about 1.1 or 1.2 mg of sodium chloride per storage container (e.g., vial).

The formulations further include sodium bicarbonate (NaHCO₃) or sodium hydrogen carbonate. Sodium bicarbonate can be present in the formulations at suitable concentrations for providing pH stability to the liquid formulation. For example, the sodium bicarbonate can be present at a concentration of about 3 mg/mL (milligrams/milliliter) or more, about 4 mg/mL or more, about 5 mg/mL or more, about 7 mg/mL or more, about 8 mg/mL or more or about 17 mg/mL or less, about 12 mg/mL or less, about 10 mg/mL or less, about 9 mg/mL or less or about 8 mg/mL or less. In other embodiment, the sodium bicarbonate can be present at a concentration of about 12 mg/mL (milligrams/milliliter) or more, about 14 mg/mL or more, about 16 mg/mL or more, or about 20 mg/mL or less, about 18 mg/mL or less or about 17 mg/mL or less. In some embodiments, the liquid injectable formulations contain a sodium bicarbonate concentration of about 4 mg/mL, about 6 mg/mL, about 7 mg/mL, about 7.5 mg/mL, about 7.6 mg/mL, about 7.7 mg/mL or about 8 mg/mL. In some embodiments, the liquid injectable formulations contain a sodium bicarbonate concentration of about 8.1 mg/mL, about 8.2 mg/mL, about 8.3 mg/mL, about 8.4 mg/mL, about 8.5 mg/mL or about 8.6 mg/mL. In one or more embodiments, the liquid lidocaine formulations contain about 4 mg to about 100 mg, about 5 mg to about 90 mg, about 6 mg to about 80 mg, about 7 mg to about 79 mg, about 7.5 mg to about 78 mg, about 7.6 mg to 77 mg, about 7.7 mg to about 76 mg or about 8 mg or 76.5 mg of sodium bicarbonate per storage container (e.g., vial). In some embodiments, the liquid injectable formulations contain a sodium bicarbonate concentration of about 12 mg/mL, about 14 mg/mL, about 15 mg/mL, about 15.2 mg/mL, about 15.4 mg/mL or about 16 mg/mL. In some embodiments, the liquid injectable formulations contain a sodium bicarbonate concentration of about 16.2 mg/mL, about 16.4 mg/mL, about 16.6 mg/mL, about 16.8 mg/mL, about 17.0 mg/mL or about 17.2 mg/mL.

In an aqueous solution of the liquid injectable formulations, bicarbonate ions are in an equilibrium state assuming a closed system such as in a container. The bicarbonate ions can generate carbon dioxide and the concentration of bicarbonate can reduce if the reaction results in excess carbon dioxide released to the headspace of a container. In this instance, the concentration of carbonate ions can increase and because carbonate ions are more alkaline as compared to the bicarbonate ions, the pH of the solution can drift and increase during storage over time and lead to unwanted instability. Lidocaine can precipitate in long term storage conditions at a pH greater than 7.5 because of its low basic solubility, pKa of 7.9, for example, as evidenced in Tables 8, 9 and 14 of the Examples. Concentration of the sodium bicarbonate buffer is adjusted to in the above amounts to provide a stable pH along with control of the amount of carbon dioxide in a headspace of a storage container, for example, carbon dioxide released by the aqueous solution of sodium bicarbonate.

The formulations can further optionally include one or more pH adjusters, for example, a single pH adjuster or a combination of two pH adjusters. The pH adjuster serves to aid in adjusting the pH of the aqueous formulation and selectively the solubilization of lidocaine or any salt thereof in the liquid formulation. The pH adjuster is used in an amount to adjust the aqueous suspension of the poorly soluble drug to a pH of about 6 to 7.5, and preferably about 7. An acid or a base is used depending on the pH of the injectable preparation of the present invention. When the injectable preparation is to be adjusted to a lower pH, an acidic pH adjuster, such as hydrochloric acid, sulfuric acid, nitric acid or acetic acid, can be used. Hydrochloric acid is preferably used. When the injectable preparation needs to be adjusted to a higher pH, a basic pH adjuster, such as sodium hydroxide, potassium hydroxide, calcium carbonate, magnesium oxide, or magnesium hydroxide, can be used. Sodium hydroxide is preferably used. Such pH adjusters can be used singly or in a combination of two or more.

In one or more embodiments, the pH adjuster is sodium hydroxide, hydrochloric acid, or a combination thereof. The pH adjuster (e.g., NaOH, HCL) can be present in the formulation at a concentration as desired to move the pH of the initially formed liquid injectable formulation to the pH range.

The pH of the formulation, prior to filling and sealing in a container, can be adjusted with other methods, either alone as a sole pH adjustment or in combination with a pH adjuster as noted above if used. The formulation, for example as a bulk liquid solution for filling into containers, can be purged with carbon dioxide gas prior to filling to reduce the pH of the bulk liquid formulation to a desirable pH for filling into containers and subsequently scaling the containers. The bulk liquid formulation can have a pH above 7.2, for example, in the range of 7.3 to 8.3, 7.4 to 8.0 or 7.5 to 7.8. To obtain a bulk liquid formulation with a pH below 7.2, and preferably below 7.0, carbon dioxide is purged and bubbled through the bulk liquid formulation to reduce the pH immediately before filling. Adjusting the pH of the liquid injectable formulation in the container, or the bulk liquid formulation used to form the liquid injectable formulation, by purging with carbon dioxide gas can be the sole step for pH adjustment or to achieve the final pH of the formulation filled into the container. The liquid injectable formulation, when the pH of the formulation is adjusted by purging with carbon dioxide gas, can be devoid of a pH adjuster ingredient as discussed above and thus carbon dioxide purging can eliminate the need for additional pH adjuster ingredients.

The liquid injectable formulations, as stored in a sealed container, can have a suitable pH in the range of about 6.5 to about 7.2 or less than 7.2. In an example, the formulation can have a pH of about 6.5 or more, about 6.6 or more, about 6.7 or more, about 6.8 or more, about 6.9 or more, or about 7.0 or more. In another example, the formulation can have a pH of about 7.2 or less, about 7.1 or less, about 7.0 or less, about 6.9 or less, about 6.8 or less, about 6.7 or less, or about 6.6 or less.

In one or more embodiments, the formulation can have a pH in the range of about 6.5 to about 7.2, about 6.6 to about 7.1, about 6.7 to about 7.0, or about 6.8 to about 7.0.

The liquid injectable lidocaine formulations containing sodium bicarbonate buffer can be free of the presence of a chelating agent (e.g., EDTA) such that no chelating agent is present or detectable in the formulation. Chelating agents can be used for achieving stability but the present invention does not rely on use of a chelating agent or stabilization agent. The liquid injectable lidocaine formulations containing sodium bicarbonate buffer can also be free of other components, such as saccharides (e.g., polysaccharides, heparin, hyaluronic acid), preservatives (e.g., parabens, methylparaben), glycols (e.g., propylene glycol, polyethylene glycol), calcium compounds or salts thereof (e.g., calcium acetate, calcium sulfate, calcium chloride, etc.), sulfates, sulfites (e.g., sodium metabisulfite), sulfides, sugars (e.g., glucose) and sugar alcohols.

In an example liquid injectable formulation, the formulation can contain lidocaine or a salt thereof in an amount of 7.8 mg/mL to 9.1 mg/mL, sodium bicarbonate as a buffering agent in an amount of about 7 to about 8 mg/mL, sodium chloride as an isotonic agent in an amount of about 6 to about 9 mg/mL, sodium hydroxide and/or hydrochloric acid as a pH adjuster in an amount as desired to adjust pH to range of about 6.5 to about 7.2, or about 6.5 to about 7.0, and a carrier (e.g., water for injection).

It is another object of this disclosure to prevent precipitation of lidocaine from the liquid formulation in the container over time as it is stored. It is desirable that the pH of the liquid formulation does not drift during storage above 7.2, and preferably remains in the range of 6.5 to 7.2. To prevent pH drift, the containers can be filled with the formulation under a blanket of carbon dioxide enriched gas to control the head space gas in the container upon sealing, for example, with a stopper. The increase of carbon dioxide gas in the head space of the container limits the dissociation of the sodium bicarbonate and release of carbon dioxide into the head space of the container, thus preventing an increase of pH over time.

The head space in the container storing the liquid formulation can contain at least 50 percent or more of carbon dioxide, with the remaining portion of the gas head space being preferably an inert gas, for example, argon or nitrogen and, ambient air. The head space can include carbon dioxide in the range of about 20 to about 95 percent, about 30 to about 90 percent, about 40 to about 90 percent, 50 to about 85 percent, about 60 to about 75 percent, or about 65, about 70 or about 75 volume percent. In other examples, the head space of the container can include 20 or more, 30 or more, 40 or more, 50 or more, 55 or more, 60 or more, 65 or more, 70 or more, 80 or more or 87 or more of volume percent of carbon dioxide.

The adjustment of the pH of the formulation before filling into the containers to below 7.2, or in the range of 6.5 to 7.0, in combination with controlling the carbon dioxide amount in the head space gas of the sealed container storing the formulation ensure a stable pH range that will not significantly increase over time. Prevention of an increase in pH reduces the likelihood of lidocaine precipitating out of the formulation and forming undesirable particulates.

In order to demonstrate the practice of the present invention, the following examples have been prepared and tested. The examples should not, however, be viewed as limiting the scope of the invention. The claims will serve to define the invention.

### EXAMPLES

### Example 1

This example demonstrates the stability of exemplary formulations including lidocaine hydrochloride, sodium bicarbonate, sodium chloride, pH adjusters and water. Bulk solution for sample preparation was formed by adding 900 g of water for injection (WFI) as 90% of the batch size. Sodium chloride was added to the WFI and resulted in a pH of 5.9. Sodium bicarbonate was further added to the solution, which resulted in the solution having a pH of 8.31. Lidocaine HCl was then added to the solution to form the bulk solution, pH of 7.50. The bulk solution was purged with carbon dioxide to reduce the pH to 6.74, dissolved oxygen (D₀)=1.7 ppm. WFI was added to the bulk solution to increase volume to 1 L, which resulted in a pH of 6.83. The bulk solution was filtered with a 0.22 μm PVDF membrane under carbon dioxide to yield a final bulk solution having a pH of 6.75.

Separate samples of the bulk solution containing the formulation of Table 1 below were prepared by filling approximately 10 mL of the bulk solution into 10 mL glass vials, tubular type 1 (normal), under carbon dioxide of 60% by volume (measured using oxygen sensor 8.4%). The vials were stoppered with a rubber stopper, Flurotec supplied by West. The sample vials did not undergo autoclaving.

TABLE 1

| Ingredient | Concentration |
|---|---|
| Lidocaine HCl | 9.09 mg/mL |
| Sodium bicarbonate | 7.64 mg/mL |
| Sodium chloride | 6.36 mg/mL |
| pH adjusted using carbon dioxide purging | pH adjust to 6.8 |
| WFI | Q.s. to 1 mL |
| Head space CO₂ | 60 volume percent |

Samples of the formulation were stored at 2-8°, 25° C./60% RH, 40° C./75% RH and 50° C./75% RH. At set periods of storage time, the samples were analyzed by HPLC and measurements of the formulations were taken as shown in Table 2 below. The HPLC conditions were as follows:

Column: ACE C18 (250×4.6 mm), 5 um; Mobile phase A: 50 mL of acid acetic glacial and 930 mL of deionized water, pH adjusted to 3.4 with NaOH 1N.; Mobile Phase B: acetonitrile (HPLC grade); Column temperature: 30° C.; Flow rate: 1.5 mL/min; Injection volume: 20 μL; Sampler temperature: 10° C.; Separation mode: gradient; Gradient program:

| Time (minutes) | % Mobile phase A | % Mobile phase B |
|---|---|---|
| 0 | 85 | 15 |
| 9 | 85 | 15 |
| 25 | 70 | 30 |
| 26 | 85 | 15 |
| 30 | 85 | 15 |

TABLE 2

| | Stor- | | Lido- | | | Total |
|---|---|---|---|---|---|---|
| Formu- | age | | caine | NaHCO₃ | Ropivacaine | Impu- |
| lation | Temp | | Assay | Assay | RCA | rities |
| Storage | (° C.) | pH | (%) | (%) | (0.2%) | (%) |
| Initial | N/A | 6.85 | 96.5 | 90.9 | ND | 0.16 |
| 1 month | 25 | 7.1 | — | 98.5 | — | — |
| | 40 | 7.2 | 99.3 | 99.5 | 0.003 | 0.07 |
| | 50 | 7.2 | 99.3 | 99.5 | 0.003 | 0.09 |
| 2 months | 2-8 | 6.9 | 96.9 | 97.5 | ND | 0.10 |
| | 40 | 7.0 | 97.4 | 97.5 | 0.004 | 0.09 |
| | 50 | 7.1 | 96.6 | 97.5 | 0.003 | 0.13 |
| 3 months | 25 | 6.9 | 97.2 | 98.5 | <LOQ | 0.05 |
| | 40 | 7.1 | 96.8 | 98.5 | <LOQ | 0.124 |
| 6 months | 25 | 7.1 | 99.3 | 98.7 | <LOQ | 0.08 |
| | 40 | 7.3 | 99.7 | 98.7 | 0.009 | 0.15 |

As can be seen from Table 2, the formulations retain 100% of the initial concentration of lidocaine hydrochloride after storage for 3 and 6 months at 25° and 40° C. The formulations further are shown to retain 100% of the initial concentration of sodium bicarbonate after storage for 3 and 6 months at 25° and 40° C. The formulations also demonstrate that no individual impurity or degradation product becomes present at about 0.05% or more or 0.03% or more after storage for 3 and 6 months at 25° and 40° C. Total impurities in the formulations remained at or below 0.15% after storage for 2, 3 and 6 months at 25° and 40° C. The results for individual impurities and total impurities as determined by HPLC and the assays for lidocaine show the formulation is stable over time under accelerated storage conditions in a pH range of 6.8 to 7.2.

The samples were also tested to determine the particulate matter present at thresholds of 10 μm and 25 μm diameter counts. The measured particulate matter data is shown in Table 3 below.

TABLE 3

| Formulation | Storage | | Particulate Matter | |
|---|---|---|---|---|
| Storage | Temp (° C.) | pH | 10 μm Counts | 25 μm Counts |
| Initial | N/A | 6.85 | 199 | 19 |
| 1 month | 25 | 7.1 | 191 | 13 |
| | 40 | 7.2 | 199 | 24 |
| | 50 | 7.2 | 189 | 28 |
| 2 months | 2-8 | 6.9 | 288 | 18 |
| | 40 | 7.0 | 124 | 16 |
| | 50 | 7.1 | 179 | 30 |
| 3 months | 25 | 6.9 | 268 | 28 |
| | 40 | 7.1 | 368 | 24 |
| 6 months | 25 | 7.1 | 459 | 27 |
| | 40 | 7.3 | 391 | 15 |

As can be seen from Table 3, the formulations exhibit pharmaceutically acceptable amounts of both 10 μm and 25 μm particulate counts after storage for 3 and 6 months at 25° and 40° C. For example, the formulations show not more than 30 and 25 or 15 particulates having an average diameter of greater than 25 μm after storage for 3 and 6 months at 25° and 40° C., respectively. The formulations also exhibit not more than 300 and 400 particulates having an average diameter of greater than 10 μm after storage for 3 months at 25° and 40° C., respectively, and not more than 500 and 400 particulates having an average diameter of greater than 10 μm after storage for 6 months at 25° and 40° C., respectively. The results for total particulates of Table 3 evidence that the formulation is stable over time under accelerated storage conditions in a pH range of 6.8 to 7.2.

### Example 2

This example demonstrates the stability of exemplary formulations including lidocaine hydrochloride, sodium bicarbonate, sodium chloride, pH adjusters and water. Separate samples containing the formulation of Table 4 below were prepared at the specified concentrations in an aqueous solution. Bulk solution for sample preparation was formed by adding 900 g of water for injection (WFI) as 90% of the batch size. Sodium chloride was added to the WFI and mixed for 10 minutes and resulted in a pH of 5.81 at 20.6° C. Sodium bicarbonate was further added to the solution and mixed for 10 minutes, which resulted in the solution having a pH of 8.31 at 19.7° C. Lidocaine HCl was then added to the solution and mixed to form the bulk solution, pH of 7.50 at 19.4° C., dissolved oxygen (D₀)=7.43 ppm. The bulk solution was purged with carbon dioxide to reduce the pH to 6.63, D₀=0.16 ppm. WFI was added to the bulk solution to increase volume to 1 L, which resulted in a pH of 6.7 at 20° C. The bulk solution was purged with carbon dioxide again with the pH remaining at 6.63 and a D₀=0.00 ppm. The bulk solution was filtered with a 0.22 μm Durapore® membrane in a glove box under carbon dioxide to yield a final bulk solution having a pH of 6.63 at 20° C. The bulk solution was divided into two lots, each with a final pH of 6.7.

In the first lot, 10 mL of the bulk solution having the formulation shown in Table 4 below was filled into two different vials. The first vials were 20 mL clear glass vials, sulfur-treated tubular type 1, supplied by Fiolax, 20 mm neck, by Schott. The second vials were 20 mL glass vials, tubular type 1 (normal). The filled vials, both sets, contained a head space of about 50%. Both vial sets were stoppered with a rubber stopper, Flurotec supplied by West. The sample vials did not undergo autoclaving.

TABLE 4

| Ingredient | Concentration |
|---|---|
| Lidocaine HCl | 9.09 mg/mL |
| Sodium bicarbonate | 7.64 mg/mL |
| Sodium chloride | 6.36 mg/mL |
| pH adjusted using carbon dioxide purging | pH adjust to 6.8 |
| WFI | Q.s. to 1 mL |
| Head space CO₂ | 75 volume percent |

The vial samples of the formulation were stored at 25° C./60% RH and 40° C./75% RH. At set periods of storage time, the samples were analyzed by HPLC, conditions as indicated above, and measurements of the formulations were taken as shown in Table 5 below.

TABLE 5

| | Stor- | | Lido- | | | |
|---|---|---|---|---|---|---|
| | age | | caine | NaHCO₃ | Ropivacaine | Total |
| | Temp | | Assay | Assay | RCA | Impu- |
| | (° C.) | pH | (%) | (%) | (0.2%) | rities |
| Formulation | | | | | | |
| (Vial 1) | | | | | | |
| Initial | N/A | 6.5 | 97.8 | 99.5 | 0.003 | 0.13 |
| 1 month | 25 | 6.5 | 98.8 | 100.7 | <LOQ | 0.123 |
| | 40 | 6.5 | 98.5 | 100.7 | <LOQ | 0.110 |
| 6 months | 25 | 6.7 | 101.9 | 100.2 | <LOQ | 0.073 |
| | 40 | 6.8 | 102.1 | 100.2 | 0.006 | 0.106 |
| Formulation | | | | | | |
| (Vial 2) | | | | | | |
| Initial | N/A | 6.8 | 98.0 | 99.5 | ND | 0.10 |
| 1 month | 25 | 6.7 | 97.8 | 100.7 | <LOQ | 0.068 |
| | 40 | 6.6 | 96.5 | 100.7 | <LOQ | 0.124 |
| 6 months | 25 | 6.7 | 101.3 | 100.2 | <LOQ | 0.075 |
| | 40 | 6.8 | 102.6 | 100.2 | <LOQ | 0.085 |

As can be seen from Table 5, the formulation in the first vials, sulfur-treated, retains 100% of the initial concentration of lidocaine and sodium bicarbonate after storage for 1 month and 6 months at 25° and 40° C. at a pH of 6.5-6.8. The formulations in the first vials also demonstrate that the individual ropivacaine impurity does not become present at about 0.01% or more after storage for 1 and 6 months at 25° and 40° C. Total impurities in the formulations remained at below 0.15% after storage for 1 month at 25° and 40° C., and at or below about 0.1% after storage for 6 months at 25° and 40° C. The results for individual impurities and total impurities as determined by HPLC and the assays for lidocaine show the formulation is stable in the first vials over time under accelerated storage conditions at a pH range of 6.5. The use of sulfur-treated vials also evidenced reduction in pH drift of the formulation, as compared to storing the same formulation in non-sulfur treated vials.

The formulation in the second vials, not sulfur-treated, also retained 100% of the initial concentration of lidocaine and sodium bicarbonate after storage for 1 month at 25° and 40° C. at a pH of 6.6-6.7. The formulations in the second vials also demonstrate that no individual impurity or degradation product becomes present at a detectable amount after storage for 1 month at 25° and 40° C. Total impurities in the formulations remained at below 0.15% after storage for 1 month at 25° and 40° C. The results for individual impurities and total impurities as determined by HPLC and the assays for lidocaine show the formulation is stable in the second vials over time under accelerated storage conditions at a pH range of 6.6-6.7.

The samples of both vials were also tested to determine the particulate matter present at thresholds of 10 μm and 25 μm diameter counts. The measured particulate matter data is shown in Table 6 below.

TABLE 6

| | Storage | | Particulate Matter | |
|---|---|---|---|---|
| | Temp (° C.) | pH | 10 μm Counts | 25 μm Counts |
| Formulation | | | | |
| (Vial 1) | | | | |
| Initial | N/A | 6.5 | — | — |
| 1 month | 25 | 6.5 | 362 | 29 |
| | 40 | 6.5 | 448 | 35 |
| 6 months | 25 | 6.7 | 431 | 33 |
| | 40 | 6.8 | 525 | 30 |
| Formulation | | | | |
| (Vial 2) | | | | |
| Initial | N/A | 6.8 | — | — |
| 1 month | 25 | 6.7 | 744 | 69 |
| | 40 | 6.6 | 849 | 96 |
| 6 months | 25 | 6.7 | 482 | 37 |
| | 40 | 6.8 | 1040 | 55 |

As can be seen from Table 6, the formulation in the first vials, sulfur-treated, exhibit pharmaceutically acceptable amounts of both 10 μm and 25 μm particulate counts after storage for 1 and 6 months at 25° and 40° C. For example, the formulations show not more than 30 and 40 particulates having an average diameter of greater than 25 μm after storage for 1 month at 25° and 40° C., respectively. In another example, the formulations show not more than 35 and 30 particulates having an average diameter of greater than 25 μm after storage for 6 months at 25° and 40° C., respectively. The formulations also exhibit not more than 400 and 500 particulates having an average diameter of greater than 10 μm after storage for 1 month at 25° and 40° C., respectively. In a further example, the formulations also exhibit not more than 450 and 550 particulates having an average diameter of greater than 10 μm after storage for 6 months at 25° and 40° C., respectively. The results for total particulates of Table 6 evidence that the formulation is stable over time under accelerated storage conditions at a pH of 6.5.

The formulation in the second vials exhibit pharmaceutically acceptable amounts of both 10 μm and 25 μm particulate counts after storage for 1 and 6 months at 25° and 40° C. The formulations show not more than 70 and 100 particulates having an average diameter of greater than 25 μm after storage for 1 month at 25° and 40° C., respectively. In another example, the formulations show not more than 40 and 55 particulates having an average diameter of greater than 25 μm after storage for 6 months at 25° and 40° C., respectively. The formulations also exhibit not more than 800 and 900 particulates having an average diameter of greater than 10 μm after storage for 1 month at 25° and 40° C., respectively. In a further example, the formulations also exhibit not more than 500 and 1050 particulates having an average diameter of greater than 10 μm after storage for 6 months at 25° and 40° C., respectively. The results for total particulates of Table 6 evidence that the formulation is stable over time under accelerated storage conditions at a pH in the range of 6.6-6.7.

### Example 3

Samples of a bulk formulation as detailed in the below table were used to evaluate the stability of the samples after multiple freeze-thaw cycles and after storage at 40° C./75% relative humidity and 2-8° C. over one month.

| Ingredient | Concentration |
|---|---|
| Lidocaine HCl | 9.09 mg/mL |
| Sodium bicarbonate | 7.64 mg/mL |
| Sodium chloride | 6.36 mg/mL |
| pH adjusted using carbon | pH adjust to 7.5 |
| dioxide purging | |
| WFI | Q.s. to 1 mL |
| Head space CO₂ | As indicated, 0, 50 and |
| | 75 volume percent |

From the bulk formulation, eleven sample lots were prepared to evaluate the stability conditions of multiple freeze-thaw cycles and storage at 40° C./75% relative humidity and 2-8° C. over one month. For each sample lot, 10 mL portions of the sample lots were filled into glass vials, tubular type 1 (normal), under carbon dioxide of 0%, 50% and 75% by volume (measured using oxygen sensor 20, 10, 5 ppm respectively). The vials were stoppered with a rubber stopper, Flurotec supplied by West. Some of the vials form sample lots underwent autoclaving prior to storage, namely vials from sample lots 9 and 11. For testing the vials 1a through 11a from the sample lots, a single freeze-thaw cycle consisted of storing the samples at −20° C. for 2 days and then transitioning the vials to storage conditions of 40° C. at 75% relative humidity for 2 days. Each vial was subjected to three individual freeze-thaw cycles. The vial formulations were observed for clarity and also tested to determine the particulate matter present at thresholds of 10 μm and 25 μm diameter counts. The initial measured particulate matter and pH data for the vials is shown in Table 7 below.

TABLE 7

| | | CO₂ % | | Initial Reading (24 h) | | | |
|---|---|---|---|---|---|---|---|
| | | head | | | | Particular | |
| | After | space | | | | Matter | |
| Sample | CO2 % | (based | Vial | De- | | Less | Less |
| Lot | purging | on O₂ | Size | scrip- | | than | than |
| Vials | in Bulk | sensor) | (mL) | tion | pH | 10 μm | 25 μm |
| 1a, 1b, 1c | — | 0% | 20 | Clear | 7.7 | 434 | 48 |
| | | | | Solution | | | |
| 2a, 2b, 2c | — | 75% | 20 | Clear | 6.9 | 1106 | 56 |
| | | | | Solution | | | |
| 3a, 3b, 3c | 7.2 | 75% | 20 | Clear | 6.9 | 682 | 38 |
| | | | | Solution | | | |
| 4a, 4b, 4c | 7.1 | 75% | 10 | Clear | 7.0 | 808 | 56 |
| | | | | Solution | | | |
| 5a, 5b, 5c | 6.9 | 75% | 20 | Clear | 6.8 | 294 | 22 |
| | | | | Solution | | | |
| 6a, 6b, 6c | 6.9 | 50% | 20 | Clear | 7.0 | 312 | 36 |
| | | | | Solution | | | |
| 7a, 7b, 7c | 6.9 | 0% | 20 | Clear | 7.2 | 408 | 58 |
| | | | | Solution | | | |
| 8a, 8b, 8c | 6.9 | 75% | 10 | Clear | 6.9 | 152 | 24 |
| | | | | Solution | | | |
| 9a, 9b, 9c | 6.8 | 75% | 20 | Clear | 6.8 | 136 | 14 |
| | | | | Solution | | | |
| 10a, 10b, | 6.6 | 75% | 20 | Clear | 6.7 | 312 | 18 |
| 10c | | | | Solution | | | |
| 11a, 11b, | 6.6 | 75% | 20 | Clear | 6.7 | 424 | 34 |
| 11c | | | | Solution | | | |

The measured particulate matter data for the vials after three individual freeze-thaw cycles is shown in Table 8 below. As can be seen from Table 7, the samples from lots 1a, 1b and 1c exhibit an initial pH of 7.7 as resulting from no carbon dioxide purging of the bulk solution and no carbon dioxide purging of the head space.

TABLE 8

| Sample | | | Particulate Matter - | Particulate Matter - |
|---|---|---|---|---|
| Lot Vial | pH | Appearance | >10 μm | >25 μm |
| 1a | 7.7 | visible particles | 1466 | 742 |
| 2a | 6.9 | clear solution | 447 | 43 |
| 3a | 6.8 | clear solution | 510 | 47 |
| 4a | 6.8 | clear solution | 743 | 42 |
| 5a | 6.7 | clear solution | 162 | 13 |
| 6a | 6.9 | clear solution | 316 | 28 |
| 7a | 7.2 | clear solution | 412 | 42 |
| 8a | 6.8 | clear solution | 257 | 7 |
| 9a | 6.7 | clear solution | 765 | 12 |
| 10a | 6.6 | clear solution | 330 | 22 |
| 11a | 6.7 | clear solution | 932 | 43 |

As can be seen from Table 8, the formulation of sample lot vials 2a-11a exhibited pharmaceutically acceptable amounts of both 10 μm and 25 μm particulate counts after three consecutive freeze-thaw cycles. For example, the vial samples show not more than 43 particulates having an average diameter of greater than 25 μm after three consecutive freeze-thaw cycles. The sample lot vials 2a-3a, 5a-8a and 10a also exhibit not more than 600 particulates having an average diameter of greater than 10 μm after three consecutive freeze-thaw cycles. The results for total particulates of Table 8 evidence that the vial formulation is stable over time after being subjected to freeze-thaw cycles at a pH of 6.6-7.2. In contrast, the formulation of sample lot 1a, which did not undergo carbon dioxide purging of the bulk solution used to the fill the vial or carbon dioxide purging the head space in the vial, exhibited visible particles. As such, the step of adjusting the pH of the bulk solution prior to filling the container, the step of purging the head space of the container with carbon dioxide, or a combination thereof results in a clear solution free of visible particles as well as the particulate characteristics discussed above.

As noted above, the lot samples were also tested in vials to determine the particulate matter present at thresholds of 10 μm and 25 μm diameter counts after one month and six months of storage at 40° C./75% relative humidity (sample lot vials 1b through 11b) and 2-8° C. (sample lot vials 1c through 11c). The initial, within 24 hours of vial filling, and one-month measured particulate matter data for sample lot vials 1b, c through 11b, c is shown in Table 9 below. All lot sample vials exhibited a clear solution upon inspection after one month.

TABLE 9

| | | | | | Partic- | Partic- | |
|---|---|---|---|---|---|---|---|
| Sam- | | | | CO₂ | ulate | ulate | |
| ple | Ini- | Vial | | % | Matter | Matter | Fi- |
| Lot | tial | Size | Storage | head | (>10 | (>25 | nal |
| Vial | pH | (mL) | Conditions | space | μm) | μm) | pH |
| 1b | 7.7 | 20 | Initial | 0 | 434 | 48 | 7.9 |
| | | | 40° C./75 RH; | | 1546 | 406 | |
| | | | 1 month | | | | |
| | | | 40° C./75 RH; | | Many | Many | 8.1 |
| | | | 6 months | | particles | particles | |
| 2b | 6.9 | 20 | Initial | 75 | 1106 | 56 | 7.1 |
| | | | 40° C./75 RH; | | 405 | 50 | |
| | | | 1 month | | | | |
| 3b | 6.9 | 20 | Initial | 75 | 682 | 38 | 6.9 |
| | | | 40° C./75 RH; | | 895 | 52 | |
| | | | 1 month | | | | |
| | | | 40° C./75 RH; | | 791 | 56 | 7.1 |
| | | | 6 months | | | | |
| 4b | 7.0 | 10 | Initial | 75 | 808 | 56 | 7.3 |
| | | | 40° C./75 RH; | | — | — | |
| | | | 1 month | | | | |
| 5b | 6.8 | 20 | Initial | 75 | 294 | 22 | 6.9 |
| | | | 40° C./75 RH; | | 298 | 28 | |
| | | | 1 month | | | | |
| | | | 40° C./75 RH; | | 410 | 46 | 6.9 |
| | | | 6 months | | | | |
| 6b | 7.0 | 20 | Initial | 50 | 312 | 36 | 7.1 |
| | | | 40° C./75 RH; | | 445 | 45 | |
| | | | 1 month | | | | |
| | | | 40° C./75 RH; | | 498 | 75 | 7.2 |
| | | | 6 months | | | | |
| 7b | 7.2 | 20 | Initial | 0 | 408 | 58 | 7.4 |
| | | | 40° C./75 RH; | | 480 | 55 | |
| | | | 1 month | | | | |
| | | | 40° C./75 RH; | | 660 | 111 | 7.6 |
| | | | 6 months | | | | |
| 8b | 6.9 | 10 | Initial | 75 | 152 | 24 | 7.0 |
| | | | 40° C./75 RH; | | 306 | 28 | |
| | | | 1 month | | | | |
| | | | 40° C./75 RH; | | 593 | 28 | 7.3 |
| | | | 6 months | | | | |
| 9b | 6.8 | 20 | Initial | 75 | 136 | 14 | 6.9 |
| | | | 40° C./75 RH; | | 603 | 20 | |
| | | | 1 month | | | | |
| | | | 40° C./75 RH; | | 525 | 15 | 7.0 |
| | | | 6 months | | | | |
| 10b | 6.7 | 20 | Initial | 75 | 312 | 18 | 6.7 |
| | | | 40° C./75 RH; | | 458 | 40 | |
| | | | 1 month | | | | |
| | | | 40° C./75 RH; | | 660 | 70 | 6.8 |
| | | | 6 months | | | | |
| 11b | 6.7 | 20 | Initial | 75 | 424 | 34 | 6.8 |
| | | | 40° C./75 RH; | | 970 | 30 | |
| | | | 1 month | | | | |
| 1c | 7.7 | 20 | Initial | 0 | 434 | 48 | 7.9 |
| | | | 2-8° C.; 1 month | | 786 | 45 | |
| 2c | 6.9 | 20 | Initial | 75 | 1106 | 56 | 7.0 |
| | | | 2-8° C.; 1 month | | 501 | 53 | |
| 3c | 6.9 | 20 | Initial | 75 | 682 | 38 | 6.8 |
| | | | 2-8° C.; 1 month | | 700 | 46 | |
| 4c | 7.0 | 10 | Initial | 75 | 808 | 56 | 7.0 |
| | | | 2-8° C.; 1 month | | 753 | 30 | |
| 5c | 6.8 | 20 | Initial | 75 | 294 | 22 | 6.8 |
| | | | 2-8° C.; 1 month | | 285 | 15 | |
| 6c | 7.0 | 20 | Initial | 50 | 312 | 36 | 6.9 |
| | | | 2-8° C.; 1 month | | 356 | 50 | |
| 7c | 7.2 | 20 | Initial | 0 | 408 | 58 | 7.2 |
| | | | 2-8° C.; 1 month | | 788 | 76 | |
| 8c | 6.9 | 10 | Initial | 75 | 152 | 24 | 6.9 |
| | | | 2-8° C.; 1 month | | 445 | 55 | |
| 9c | 6.8 | 20 | Initial | 75 | 136 | 14 | 6.8 |
| | | | 2-8° C.; 1 month | | 336 | 11 | |
| 10c | 6.7 | 20 | Initial | 75 | 312 | 18 | 6.7 |
| | | | 2-8° C.; 1 month | | 530 | 40 | |
| 11c | 6.7 | 20 | Initial | 75 | 424 | 34 | 6.8 |
| | | | 2-8° C.; 1 month | | 498 | 35 | |

As can be seen from Table 9, the formulation of sample lot vial 1b exhibited more than a 3.5 fold increase in particulates having an average diameter of greater than 10 μm and nearly a 8.5 fold increase in particulates having an average diameter of greater than 25 μm when the carbon dioxide volume percent in the vial headspace was 0% and the pH was 7.7 or above at accelerated aging conditions of 40° C./75% relative humidity. Similar to the formulation of sample lot vial 1a of Table 8, which also did not undergo carbon dioxide purging of the bulk solution used to the fill the vial or carbon dioxide purging the head space in the vial, the formulation of sample lot vial 1b exhibited visible particles after storage for 6 months at 40° C./75% RH. As such, the step of adjusting the pH of the bulk solution prior to filling the container, the step of purging the head space of the container with carbon dioxide, or a combination thereof as used in the remaining sample lot vials results in a clear solution free of visible particles as well as the particulate characteristics discussed below for at least 1 and 6 months at 40° C./75% RH. The pH of the formulation of sample lot vial 1b also exhibited a pH increase to 8.1 after storage for 6 months at 40° C./75% RH. In contrast, the pH of the formulations of the remaining sample lot vials, which were at least subjected to carbon dioxide purging the bulk solution, carbon dioxide purging the head space in the vial, or a combination thereof, maintained a pH at or below 7.6 after storage for 6 months at 40° C./75% RH which evidenced a result of a clear solution free of visible particles.

The remaining sample lot vials stored at 40° C./75% relative humidity exhibited acceptable amounts of both 10 μm and 25 μm particulate counts. For example, the formulations of sample lot vials 2b-11b show not more than 55 particulates having an average diameter of greater than 25 μm after storage for 1 month at 40° C./75% RH. After storage for 6 months at 40° C./75% RH, the formulations of sample lot vials 3b and 5b-10b show not more than 115 particulates having an average diameter of greater than 25 μm, and selectively the formulations of sample lot vials 3b, 5b-6b and 8b-10b show not more than 75 particulates having an average diameter of greater than 25 μm. The formulations, 2b-11b, also exhibited not more than 970 particulates having an average diameter of greater than 10 μm after storage for 1 month at 40° C./75% RH. In another example, after storage for 6 months at 40° C./75% RH, the formulations of sample lot vials 3b and 5b-10b exhibited not more than 800 particulates having an average diameter of greater than 10 μm.

At cold storage conditions of 2-8° C. for one month, sample lot vials 1c-11c exhibited not more than 76 particulates having an average diameter of greater than 25 μm after storage for 1 month at 2-8° C. The largest count of particulates having an average diameter of greater than 25 μm occurred in sample lot vial 7c having a carbon dioxide volume percent in the vial headspace of 0%. The sample lot formulations also exhibited not more than 788 particulates having an average diameter of greater than 10 μm after storage for 1 month at 2-8° C. Again, the largest count of particulates having an average diameter of greater than 10 μm occurred in sample lot vial 7c having a carbon dioxide volume percent in the vial headspace of 0%. Samples 2, 4 and 5, which had a pH range of 6.8-7.0 with 75% CO₂ vial headspace, exhibited decreases in particulates having an average diameter of greater than 25 μm and 10 μm after storage for 1 month at 2-8° C.

### Example 4

This example demonstrates the stability of exemplary formulations including lidocaine hydrochloride, sodium bicarbonate, sodium chloride, pH adjusters and water. The bulk solution of the formulation was separated into seven different lots and purged with carbon dioxide to reduce the pH to form seven different lots of bulk solution that were used to fill vials. The bulk solution for formulations 1-4 were adjusted to 7, formulation 5 was adjusted to 6.8, formulation 6 was adjusted to 7.3 and formulation 7 was adjusted to 7.5. Separate samples of the bulk solution containing the formulation of Table 10 below were prepared by filling approximately 10 mL of the bulk solution into 20 mL glass vials, tubular type 1 (normal), under carbon dioxide of 20, 40, 60 or 75% by volume (measured using oxygen sensor 16.8, 12.6, 8.4 or 5.2%). The vials were stoppered with a butyl rubber stopper, Flurotec supplied by West. The sample vials did not undergo autoclaving.

TABLE 10

| Ingredient | Concentration |
|---|---|
| Lidocaine HCl | 10.0 mg/mL |
| Sodium bicarbonate | 8.4 mg/mL |
| Sodium chloride | 1.0 mg/mL |
| pH adjusted using carbon | pH adjust to as indicated for Forms. 1-7 |
| dioxide purging | |
| WFI | Q.s. to 1 mL |
| Head space CO₂ | As indicated, 20, 40, 60 and 75 volume |
| | percent |

The vial samples of the formulation were stored at 25° C./60% RH and 40° C./75% RH. At set periods of storage time, the samples were analyzed by HPLC, conditions as indicated above, and measurements of the formulations were taken as shown in Table 11 below. During testing, all vials were clear solutions and free of particles.

TABLE 11

| | | CO₂ % | | Lidocaine | NaHCO₃ | Ropivacaine | Total |
|---|---|---|---|---|---|---|---|
| | Storage Temp | head | | Assay | Assay | RCA | Impurities |
| Form. | (° C.)/Time | space | pH | (%) | (%) | (0.2%) | (%) |
| 1 | Initial | 20 | 7 | 98.3 | 100.5 | Not | 0.02 |
| | | | | | | detected | |
| | 40° C./75 | | 7.3 | 99.6 | 100.4 | <LOQ | 0.04 |
| | RH; 1 month | | | | | | |
| | 40° C./75 | | 7.2 | 99.8 | 100.4 | 0.005 | 0.03 |
| | RH; 2 months | | | | | | |
| | 25° C./60 | | 7.1 | 98.6 | 98.4 | Not | 0.03 |
| | RH; 3 months | | | | | detected | |
| | 40° C./75 | | 7.2 | 97.5 | 97.4 | 0.009 | 0.06 |
| | RH; 3 months | | | | | | |
| 2 | Initial | 40 | 7 | 100.1 | 100.5 | Not | 0.02 |
| | | | | | | detected | |
| | 40° C./75 | | 7.1 | 100.3 | 100.4 | <LOQ | 0.04 |
| | RH; 1 month | | | | | | |
| | 40° C./75 | | 7.1 | 99.5 | 100.4 | <LOQ | 0.01 |
| | RH; 2 months | | | | | | |
| | 25° C./60 | | 7.0 | 100.2 | 100.4 | Not | 0.02 |
| | RH; 3 months | | | | | detected | |
| | 40° C./75 | | 7.0 | 100.0 | 100.4 | 0.01 | 0.04 |
| | RH; 3 months | | | | | | |
| 3 | Initial | 60 | 6.9 | 100 | 100.5 | Not | 0.02 |
| | | | | | | detected | |
| | 40° C./75 | | 7.2 | 100.1 | 100.4 | <LOQ | 0.03 |
| | RH; 1 month | | | | | | |
| | 40° C./75 | | 7 | 99.8 | 100.4 | <LOQ | 0.02 |
| | RH; 2 months | | | | | | |
| | 25° C./60 | | 6.9 | 99.5 | 100.4 | Not | 0.02 |
| | RH; 3 months | | | | | detected | |
| | 40° C./75 | | 6.9 | 100.1 | 100.4 | 0.005 | 0.03 |
| | RH; 3 months | | | | | | |
| 4 | Initial | 75 | 6.8 | 99.5 | 100.5 | Not | 0.02 |
| | | | | | | detected | |
| | 40° C./75 | | 7 | 100 | 100.4 | <LOQ | 0.03 |
| | RH; 1 month | | | | | | |
| | 40° C./75 | | 7 | 99.9 | 99.4 | <LOQ | 0.02 |
| | RH; 2 months | | | | | | |
| | 25° C./60 | | 6.8 | 100.4 | 100.4 | Not | 0.02 |
| | RH; 3 months | | | | | detected | |
| | 40° C./75 | | 6.9 | 100.5 | 100.4 | <LOQ | 0.02 |
| | RH; 3 months | | | | | | |
| 5 | Initial | 60 | 6.8 | 99 | 100.5 | Not | 0.03 |
| | | | | | | detected | |
| | 40° C./75 | | 6.9 | 99.4 | 100.4 | <LOQ | 0.03 |
| | RH; 1 month | | | | | | |
| | 40° C./75 | | 7 | 99.9 | 100.4 | <LOQ | 0.02 |
| | RH; 2 months | | | | | | |
| | 25° C./60 | | 6.7 | 101.8 | 100.4 | Not | 0.02 |
| | RH; 3 months | | | | | detected | |
| | 40° C./75 | | 6.9 | 101.3 | 100.4 | 0.005 | 0.04 |
| | RH; 3 months | | | | | | |
| 6 | Initial | 60 | 7 | 100.7 | 100.5 | Not | 0.03 |
| | | | | | | detected | |
| | 40° C./75 | | 7.1 | 99.6 | 100.4 | <LOQ | 0.03 |
| | RH; 1 month | | | | | | |
| | 40° C./75 | | 7.3 | 100 | 101.4 | <LOQ | 0.01 |
| | RH; 2 months | | | | | | |
| | 25° C./60 | | 7.0 | 100.9 | 100.4 | Not | 0.02 |
| | RH; 3 months | | | | | detected | |
| | 40° C./75 | | 7.1 | 99.9 | 100.4 | 0.006 | 0.05 |
| | RH; 3 months | | | | | | |
| 7 | Initial | 60 | 6.9 | 99.7 | 99.5 | Not | 0.02 |
| | | | | | | detected | |
| | 40° C./75 | | 7.1 | 99.6 | 100.4 | <LOQ | 0.04 |
| | RH; 1 month | | | | | | |
| | 40° C./75 | | 7.3 | 99.6 | 101.4 | <LOQ | 0.02 |
| | RH; 2 months | | | | | | |
| | 25° C./60 | | 7.0 | 101.2 | 100.4 | Not | 0.03 |
| | RH; 3 months | | | | | detected | |
| | 40° C./75 | | 7.1 | 98.7 | 100.4 | 0.006 | 0.03 |
| | RH; 3 months | | | | | | |

As can be seen from Table 11, the formulations retain 99-100% of the initial concentration of lidocaine hydrochloride after storage for 1, 2 and 3 months at 25° C./60 RH and 40° C./75 RH. The formulations further are shown to retain 99-100% of the initial concentration of sodium bicarbonate after storage for 1, 2 and 3 months at 25° C./60 RH and 40° C./75 RH. The formulations also demonstrate that no individual impurity or degradation product becomes present at 0.01% or more after storage for 1, 2 and 3 months at 25° C./60 RH and 40° C./75 RH and at a carbon dioxide headspace of 20, 40, 60 or 75% by volume and pH in the range of 6.8-7.3. Total impurities in the formulations remained at or below 0.05% or 0.06% after storage for 1, 2 and 3 months at 25° C./60 RH and 40° C./75 RH and at a carbon dioxide headspace of 20, 40, 60 or 75% by volume and pH in the range of 6.8-7.3. The results for individual impurities and total impurities as determined by HPLC and the assays for lidocaine show the formulation is stable over time under accelerated storage conditions in a pH range of 6.8 to 7.3 and at a carbon dioxide headspace of 20, 40, 60 or 75% by volume.

The samples were also tested to determine the particulate matter present at thresholds of 10 μm and 25 μm diameter counts. The measured particulate matter data is shown in Table 12 below.

TABLE 12

| | Storage | | CO₂ % | Particulate Matter | |
|---|---|---|---|---|---|
| Formu- | Temp | | head | 10 μm | 25 μm |
| lation | (° C.)/Time | pH | space | Counts | Counts |
| 1 | Initial | 7 | 20 | 79 | 6 |
| | 40° C./75 RH; | 7.3 | | 88 | 7 |
| | 1 month | | | | |
| | 40° C./75 RH; | 7.2 | | 147 | 21 |
| | 2 months | | | | |
| | 25° C./60 RH; | 7.1 | | 89 | 9 |
| | 3 months | | | | |
| | 40° C./75 RH; | 7.2 | | 92 | 12 |
| | 3 months | | | | |
| 2 | Initial | 7 | 40 | 39 | 5 |
| | 40° C./75 RH; | 7.1 | | 144 | 8 |
| | 1 month | | | | |
| | 40° C./75 RH; | 7.1 | | 73 | 11 |
| | 2 months | | | | |
| | 25° C./60 RH; | 7.0 | | 55 | 6 |
| | 3 months | | | | |
| | 40° C./75 RH; | 7.0 | | 54 | 3 |
| | 3 months | | | | |
| 3 | Initial | 6.9 | 60 | 216 | 30 |
| | 40° C./75 RH; | 7.2 | | 67 | 6 |
| | 1 month | | | | |
| | 40° C./75 RH; | 7 | | 77 | 9 |
| | 2 months | | | | |
| | 25° C./60 RH; | 6.9 | | 44 | 3 |
| | 3 months | | | | |
| | 40° C./75 RH; | 6.9 | | 44 | 4 |
| | 3 months | | | | |
| 4 | Initial | 6.8 | 75 | 34 | 4 |
| | 40° C./75 RH; | 7 | | 33 | 2 |
| | 1 month | | | | |
| | 40° C./75 RH; | 7 | | 162 | 15 |
| | 2 months | | | | |
| | 25° C./60 RH; | 6.8 | | 55 | 2 |
| | 3 months | | | | |
| | 40° C./75 RH; | 6.9 | | 131 | 4 |
| | 3 months | | | | |
| 5 | Initial | 6.8 | 60 | 166 | 16 |
| | 40° C./75 RH; | 6.9 | | 133 | 11 |
| | 1 month | | | | |
| | 40° C./75 RH; | 7 | | 169 | 5 |
| | 2 months | | | | |
| | 25° C./60 RH; | 6.7 | | 98 | 9 |
| | 3 months | | | | |
| | 40° C./75 RH; | 6.9 | | 140 | 9 |
| | 3 months | | | | |
| 6 | Initial | 7 | 60 | 199 | 12 |
| | 40° C./75 RH; | 7.1 | | 115 | 5 |
| | 1 month | | | | |
| | 40° C./75 RH; | 7.3 | | 583 | 103 |
| | 2 months | | | | |
| | 25° C./60 RH; | 7.0 | | 113 | 5 |
| | 3 months | | | | |
| | 40° C./75 RH; | 7.1 | | 152 | 4 |
| | 3 months | | | | |
| 7 | Initial | 6.9 | 60 | 88 | 6 |
| | 40° C./75 RH; | 7.1 | | 106 | 3 |
| | 1 month | | | | |
| | 40° C./75 RH; | 7.3 | | 97 | 9 |
| | 2 months | | | | |
| | 25° C./60 RH; | 7.0 | | 69 | 5 |
| | 3 months | | | | |
| | 40° C./75 RH; | 7.1 | | 108 | 3 |
| | 3 months | | | | |

As can be seen from Table 12, the formulations exhibit pharmaceutically acceptable amounts of both 10 μm and 25 μm particulate counts after storage for 1, 2 and 3 months at 25° C./60 RH and 40° C./75 RH. For example, the formulations 1-5 and 7 show not more than 30 particulates having an average diameter of greater than 25 μm after storage for 1 month and 2 months at 40° C./75 RH and at a carbon dioxide headspace of 20, 40, 60 or 75% by volume, respectively. The formulations, 1-5 and 7, also exhibit not more than 175 particulates having an average diameter of greater than 10 μm after storage for 1 and 2 months at 40° C./75 RH and at a carbon dioxide headspace of 20, 40, 60 or 75% by volume, respectively. After 3 months of storage at 40° C./75 RH, all formulations exhibit not more than 152 particulates having an average diameter of greater than 10 μm and not more than 12 particulates having an average diameter of greater than 25 μm. The results for total particulates of Table 12 evidence that the formulation is stable over time under accelerated storage conditions at a pH of 6.8-7.3.

### Example 5

Exemplary formulations including lidocaine hydrochloride, sodium bicarbonate, sodium chloride, pH adjusters and water were formed as shown in Table 13 below. The bulk solution was separated into two lots, A and B. Lot A was not purged with carbon dioxide to reduce the pH, while Lot B was purged with carbon dioxide to reduce the pH before the bulk solution was used to fill vials.

The vials of Lots A and B were prepared by filling approximately 10 mL of the bulk solution into 20 mL glass vials, Type 1 molded, under carbon dioxide of 0 or 75% by volume (measured using oxygen sensor 21 or 5.2%). The vials were stoppered with a butyl rubber stopper, Flurotec supplied by West. The sample vials did undergo autoclaving.

TABLE 13

| Ingredient | Concentration |
|---|---|
| Lidocaine HCl | 10.0 mg/mL |
| Sodium bicarbonate | 8.4 mg/mL |
| Sodium chloride | 7.0 mg/mL |
| pH adjusted using carbon | As indicated |
| dioxide purging | |
| WFI | Q.s. to 1 mL |
| Head space CO₂ | As indicated, 0 and |
| | 75 volume percent |

The samples of both vials were also tested to determine the particulate matter present at thresholds of 10 μm and 25 μm diameter counts. The measured particulate matter data is shown in Table 14 below.

TABLE 14

| | | CO₂ % | | Particulate Matter | |
|---|---|---|---|---|---|
| | Storage | head | | 10 μm | 25 μm |
| | Temp (° C.) | space | pH | Counts | Counts |
| Formulation | | | | | |
| (Lot A) | | | | | |
| Initial | N/A | 0 | 7.8 | 921 | 22 |
| 1 month | 2-8 | 0 | 7.9 | 623 | 18 |
| | 40 / 75% | 0 | 8.1 | 5846 (visible | 1543 (visible |
| | RH | | | crystalline | crystalline |
| | | | | particles) | particles |
| 6 months | 40 / 75% | 0 | 8.1 | Error test - | Error test - |
| | RH | | | Visible | Visible |
| | | | | crystalline | crystalline |
| | | | | particles | particles |
| Formulation | | | | | |
| (Lot B) | | | | | |
| Initial | N/A | 75 | 6.8 | 416 | 33 |
| 1 month | 2-8 | 75 | 6.8 | 838 | 18 |
| | 40 / 75% | 75 | 6.9 | 623 | 60 |
| | RH | | | | |
| 6 months | 40 / 75% | 75 | 7.1 | 556 (clear | 38 (clear |
| | RH | | | solution) | solution) |

As seen in Table 14, the absence of carbon dioxide in the head space resulted in an initial pH of around 7.8 and an increase in pH of the solution over 1 and 6 months. After 1 month of storage at 40° C. and 75% relative humidity, the vials with no carbon dioxide in the head space exhibited visible crystalline particles (visible particles began to form after 1-2 weeks) and had more than 1500 particulates having an average diameter of greater than 25 μm and more than 5800 particulates having an average diameter of greater than 10 μm. After 6 months of storage at 40° C. and 75% relative humidity, the vials with no carbon dioxide in the head space exhibited large quantities of visible particles. The vials of Lot A exhibited a pH drift upward to a value of 8.1 over the storage period that impacted precipitation of particulates as evidenced by the appearance of visible particles. This further evidences the impact that carbon dioxide purging and/or presence in the vial headspace has on stability and particulate formation in the injectable solutions. In contrast, after 1 month of storage at 40° C. and 75% relative humidity, the vials having 75% carbon dioxide in the headspace measured not more than 60 particles having an average diameter of greater than 25 μm and not more than 623 particulates having an average diameter of greater than 10 μm. This represents a 89% decrease (10 μm particles) and a 96% decrease (25 μm particles) as compared to vials from Lot A. Notably, the vials from Lot B exhibited a clear solution during the entire test and storage period and a decrease in particles at 6 months as compared to measurements at 1 month.

### Example 6

Exemplary formulations including lidocaine hydrochloride, sodium bicarbonate, sodium chloride, pH adjuster and water were formed as shown in Table 15 below. The bulk solution was separated into one lot—Lot A. Lot A was not purged with carbon dioxide to reduce the pH, but rather hydrochloric acid was used to adjust pH before the bulk solution was used to fill vials.

The vials of Lots A were prepared by filling approximately 10 mL of the bulk solution into 10 mL glass vials, Type 1 molded, under carbon dioxide of 0% by volume(measured using oxygen sensor 21%). The vials were stoppered with a butyl rubber stopper, Flurotec supplied by West. The sample vials did undergo autoclaving.

TABLE 15

| Ingredient | Concentration |
|---|---|
| Lidocaine HCl | 9.09 mg/mL |
| Sodium bicarbonate | 7.64 mg/mL |
| Sodium chloride | 6.36 mg/mL |
| pH adjusted using hydrochloric acid | Q.s. to pH |
| WFI | Q.s. to 1 mL |
| Head space CO₂ | None |

The samples of the vials were also tested to determine the particulate matter present at thresholds of 10 μm and 25 μm diameter counts. The measured particulate matter data is shown in Table 16 below.

TABLE 16

| | | CO₂ % | | Particulate Matter | |
|---|---|---|---|---|---|
| Formulation | Storage | head | | 10 μm | 25 μm |
| (Lot A) | Temp (° C.) | space | pH | Counts | Counts |
| Initial | N/A | 0 | 7.3 | 790 (clear | 56 (clear |
| | | | | solution) | solution) |
| 1 month | 2-8 | 0 | | 358 (clear | 16 (clear |
| | | | | solution) | solution) |
| | 40 / 75% | 0 | 7.2 | 471 (visible | 21 (visible |
| | RH | | | small | small |
| | | | | particles | particles |
| | | | | formed) | formed) |
| 6 months | 40 / 75% | 0 | 7.6 | 1063 (visible | 96 (visible |
| | RH | | | small | small |
| | | | | particles | particles |
| | | | | formed) | formed) |

As seen in Table 16, the absence of carbon dioxide in the head space and use thereof to adjust the initial pH of the bulk resulted in the presence of visible small particles before 1 month of storage at elevated temperature. After 1 month of storage at 40° C. and 75% relative humidity, the vials exhibited visible (visible particles began to form after 2 weeks) and had more than 1050 particulates having an average diameter of greater than 10 μm at 6 months.

### Example 7

Exemplary formulations including lidocaine hydrochloride, sodium bicarbonate, sodium chloride, pH adjusters and water were formed as shown in Table 17 below. The bulk solution was separated into two lots, A and B. Lot A was not purged with carbon dioxide to reduce the pH, while Lot B was purged with carbon dioxide to reduce the pH to 6.9 before the bulk solution was used to fill vials. The bulk solution of Lot A, which did not undergo carbon dioxide purging, showed particle precipitation after formation and thus no vials were filled for a stability study.

The vials of Lot B were prepared by filling approximately 10 mL of the bulk solution into 10 mL glass vials, Type 1 molded, under carbon dioxide of 75% by volume (measured using oxygen sensor 5.2%). The vials were stoppered with a butyl rubber stopper, Flurotec supplied by West. The sample vials did undergo autoclaving.

TABLE 17

| Ingredient | Concentration |
|---|---|
| Lidocaine HCl | 18.18 mg/mL |
| Sodium bicarbonate | 7.64 mg/mL |
| Sodium chloride | 6.40 mg/mL |
| pH adjusted using carbon dioxide purging | 6.9 |
| WFI | Q.s. to 1 mL |
| Head space CO₂ | 75 volume percent |

The samples of the Lot B vials were tested to determine the particulate matter present at thresholds of 10 μm and 25 μm diameter counts. The measured particulate matter data is shown in Table 18 below.

TABLE 18

| | | CO₂ % | | Particulate Matter | |
|---|---|---|---|---|---|
| Formulation | Storage | head | | 10 μm | 25 μm |
| (Lot B) | Temp (° C.) | space | pH | Counts | Counts |
| Initial | N/A | 75 | 6.8 | 1093 (clear | 33 (clear |
| | | | | solution) | solution) |
| 1 month | 2-8 | 75 | 6.7 | 591 (clear | 30 (clear |
| | | | | solution) | solution) |
| 3 months | 40 / 75% | 75 | 6.9 | 800 (clear | 43 (clear |
| | RH | | | solution) | solution) |

As seen in Table 18, the carbon dioxide in the head space and a pH in the range of 6.7-6.9 resulted in a clear solution after 3 months at 40° C. and 75% relative humidity even at a lidocaine concentration over 18 mg/mL. After 3 months of storage at 40° C. and 75% relative humidity, the vials having 75% carbon dioxide in the headspace measured not more than 45 particles having an average diameter of greater than 25 μm and not more than 800 particulates having an average diameter of greater than 10 μm. Notably, the vials from Lot B exhibited a clear solution during the entire test and storage period up to over 6 months.

### Example 8

Exemplary formulations including lidocaine hydrochloride, sodium bicarbonate, sodium chloride, pH adjusters and water were formed as shown in Table 19 below. The bulk solution was purged with carbon dioxide to reduce the pH to 6.9 before the bulk solution was used to fill vials into two lots, A and B. The vials of Lots A and B were prepared by filling approximately 10 mL of the bulk solution into 10 mL glass vials, Type 1 molded, under carbon dioxide of 20 or 40% by volume (measured using oxygen sensor 16.8% and 12.6%). The vials were stoppered with a butyl rubber stopper, Flurotec supplied by West. The sample vials did undergo autoclaving.

TABLE 19

| Ingredient | Concentration |
|---|---|
| Lidocaine HCl | 9.09 mg/mL |
| Sodium bicarbonate | 7.64 mg/mL |
| Sodium chloride | 6.36 mg/mL |
| pH adjusted using carbon | 6.9 |
| dioxide purging | |
| WFI | Q.s. to 1 mL |
| Head space CO₂ | As indicated, 20 and 40 volume percent |

The samples of both vial lots were also tested to determine the particulate matter present at thresholds of 10 μm and 25 μm diameter counts. The measured particulate matter data is shown in Table 20 below.

TABLE 20

| | | CO₂ % | | Particulate Matter | |
|---|---|---|---|---|---|
| | Storage | head | | 10 μm | 25 μm |
| | Temp (° C.) | space | pH | Counts | Counts |
| Formulation | | | | | |
| (Lot A) | | | | | |
| Initial | N/A | 20 | 7.0 | 900 (clear | 43 (clear |
| | | | | solution) | solution) |
| 2 weeks | 2-8 | 20 | 7.0 | 1368 (clear | 33 (clear |
| | | | | solution) | solution) |
| 1 month | 40 / 75% | 20 | 7.1 | 1593 (clear | 56 (clear |
| | RH | | | solution) | solution) |
| 3 months | 40 / 75% | 20 | 7.2 | 1035 (clear | 81 (clear |
| | RH | | | solution) | solution) |
| 6 months | 40 / 75% | 20 | 7.3 | 3063 (clear | 108 (clear |
| | RH | | | solution) | solution) |
| Formulation | | | | | |
| (Lot B) | | | | | |
| Initial | N/A | 40 | 6.9 | 660 (clear | 43 (clear |
| | | | | solution) | solution) |
| 2 weeks | 2-8 | 40 | 6.9 | 833 (clear | 31 (clear |
| | | | | solution) | solution) |
| 1 month | 40 / 75% | 40 | 7.0 | 1356 (clear | 43 (clear |
| | RH | | | solution) | solution) |
| 3 months | 40 / 75% | 40 | 7.1 | 769 (clear | 65 (clear |
| | RH | | | solution) | solution) |
| 6 months | 40 / 75% | 40 | 7.2 | 708 (clear | 16 (clear |
| | RH | | | solution) | solution) |

As seen in Table 20, the lower concentration (20%) of carbon dioxide in the head space resulted in a greater concentration of particles in the solution over storage of 2 weeks to 6 months at both storage conditions, 2-8° C. and 40° C. and 75% relative humidity. After 2 weeks of storage at 2-8° C., the vials with 20% carbon dioxide in the head space had 1368 particulates having an average diameter of greater than 10 μm whereas the vials with 40% carbon dioxide in the head space had 833 particulates having an average diameter of greater than 10 μm, which represents a 39% decrease in particulates. In another example, after 6 months of storage at 40° C. and 75% relative humidity, the vials with 20% carbon dioxide in the head space had 3063 and 108 particulates having an average diameter of greater than 10 μm and 25 μm, respectively, whereas the vials with 40% carbon dioxide in the head space had 708 and 16 particulates having an average diameter of greater than 10 μm and 25 μm, respectively, which represents a 77% and 85% decrease in particulates.

### Example 9

This example demonstrates the stability of exemplary formulations including lidocaine hydrochloride, sodium bicarbonate, sodium chloride, pH adjusters and water. The bulk solution was purged with carbon dioxide to reduce the pH to form four different lots of bulk solution that were used to fill vials, Lots A, B, C1, C2 and D. The bulk solution for Lot A was adjusted to 7.4, Lot B was adjusted to 7.2, Lots C1 and C2 were adjusted to 7 and Lot D was adjusted to 6.8.

Separate samples of the bulk solution from each lot containing the formulation of Table 21 below were prepared by filling approximately 10 mL of the bulk solution into 10 mL glass vials, Type 1 molded, under carbon dioxide of 40 or 75% by volume (measured using oxygen sensor 12.6 or 5.2%). The vials were stoppered with a rubber stopper, Omniflex plus. The sample vials did undergo autoclaving.

TABLE 21

| Ingredient | Concentration |
|---|---|
| Lidocaine HCl | 9.09 mg/mL |
| Sodium bicarbonate | 7.64 mg/mL |
| Sodium chloride | 6.36 mg/mL |
| pH adjusted using carbon | As indicated, 6.8-7.4 |
| dioxide purging | |
| WFI | Q.s. to 1 mL |
| Head space CO₂ | As indicated, 40 and 75 volume percent |

The samples were also tested to determine the particulate matter present at thresholds of 10 μm and 25 μm diameter counts. The measured particulate matter data is shown in Table 22 below.

TABLE 22

| | | | CO₂ % | Particulate Matter | |
|---|---|---|---|---|---|
| | Storage Temp | | head | 10 μm | 25 μm |
| Formulation | (° C.) / Time | pH | space | Counts | Counts |
| Lot A (bulk | Initial | 6.8 | 75 | 362 | 27 |
| pH 7.4) | 40° C./ 75 RH; | 7.1 | | 499 | 27 |
| | 1 month | | | | |
| | 40° C./ 75 RH; | 7.1 | | 135 | 5 |
| | 2 months | | | | |
| | 40° C./ 75 RH; | 7.1 | | 590 | 32 |
| | 3 months | | | | |
| Lot B (bulk | Initial | 7.0 | 75 | 224 | 14 |
| pH 7.2) | 40° C./ 75 RH; | 7.1 | | 1506 | 31 |
| | 1 month | | | | |
| | 40° C./ 75 RH; | 7.2 | | 42 | 2 |
| | 2 months | | | | |
| | 40° C./ 75 RH; | 7.5 | | 461 | 8 |
| | 3 months | | | | |
| Lot C1 (bulk | Initial | 6.9 | 40 | 283 | 15 |
| pH 7.0) | 40° C./ 75 RH; | 7.2 | | 188 | 9 |
| | 1 month | | | | |
| | 40° C./ 75 RH; | 7.4 | | 131 | 7 |
| | 2 months | | | | |
| | 40° C./ 75 RH; | 7.5 | | 166 | 12 |
| | 3 months | | | | |
| Lot C2 (bulk | Initial | 7.0 | 75 | 220 | 9 |
| pH 7.0) | 40° C./ 75 RH; | 7.1 | | 1750 | 49 |
| | 1 month | | | | |
| | 40° C./ 75 RH; | 7.2 | | 105 | 8 |
| | 2 months | | | | |
| | 40° C./ 75 RH; | 7.2 | | 640 | 9 |
| | 3 months | | | | |
| Lot D (bulk | Initial | 6.8 | 75 | 366 | 30 |
| pH 6.8) | 40° C./ 75 RH; | 7.0 | | 479 | 17 |
| | 1 month | | | | |
| | 40° C./ 75 RH; | 7.2 | | 78 | 4 |
| | 2 months | | | | |
| | 40° C./ 75 RH; | 7.1 | | 170 | 8 |
| | 3 months | | | | |

All solutions listed in Table 22 were clear during testing. As can be seen from the data, adjusting the pH of bulk solution in the range of 6.8 to 7.2 with carbon dioxide purging prior to filling vials, coupled with a carbon dioxide content in the vial head space at or above 40%, can control particulate matter formation. After 3 months of storage at 40° C. and 75% relative humidity, the vials filled with a bulk solution adjusted to pH of 7.4 had the highest concentration of particulates, 32, having an average diameter of greater than 25 μm as compared to the closest amount of 12 for the only vials not having a 75% carbon dioxide headspace, which represents a reduction of 63%. Lot A nevertheless represents a formulation that is stable and clear over time under accelerated storage conditions when the bulk solution is adjusted to a pH of 7.4 with carbon dioxide before filling.

Many variations and modifications may be made to the above-described embodiments of the disclosure without departing substantially from the spirit and various principles of the disclosure. All such modifications and variations are intended to be included herein within the scope of this disclosure and protected by the following claims.

1. An injectable liquid formulation in a container, the formulation comprising:
a. lidocaine or a pharmaceutically acceptable salt thereof present at a concentration of about 5 to about 20 mg/mL as the sole pharmaceutically active agent;
b. sodium bicarbonate buffer present at a concentration of about 4 to about 17 mg/mL; and
c. a carrier,
wherein the injectable liquid formulation has a pH of about 6.5 to about 7.4 and the container comprises a gas headspace, the gas headspace comprises greater than 20% carbon dioxide.

2. The injectable liquid formulation of claim 1, wherein the pharmaceutically acceptable salt of lidocaine is selected from the group consisting of lidocaine hydrochloride, lidocaine hydrobromide, lidocaine oxalate, lidocaine fumarate, lidocaine adipate, lidocaine maleate, lidocaine malonate and lidocaine tosylate.

3. The injectable liquid formulation of claim 2, wherein the pharmaceutically acceptable salt of lidocaine is present at a concentration of about 5 mg/mL, about 9 mg/mL, about 10 mg/mL or about 20 mg/mL.

4. The injectable liquid formulation of claim 1, wherein the weight ratio of the sodium bicarbonate buffer to the lidocaine or a pharmaceutically acceptable salt thereof is in the range of about 0.3:1 to about 1.4:1.

5. The injectable liquid formulation of claim 2, wherein the weight ratio of the sodium bicarbonate buffer to the pharmaceutically acceptable salt of lidocaine is about 0.84:1.

6. The injectable liquid formulation of claim 1, wherein the sodium bicarbonate is the sole buffer in the injectable liquid formulation.

7. The injectable liquid formulation of claim 1, the formulation further comprising sodium chloride present at a concentration of about 1 to about 8 mg/mL.

8. The injectable liquid formulation of claim 1, the formulation further comprising a pH adjuster.

9. The injectable liquid formulation of claim 1, wherein the gas head space comprises 40% or more of carbon dioxide.

10. The injectable liquid formulation of claim 9, wherein the container has a sealed internal volume and the gas head space comprises between 10 and 60 percent of the sealed internal volume of the container.

11. The injectable liquid formulation of claim 1, wherein the injectable liquid formulation has a pH of about 6.7 to about 7.2.

12. The injectable liquid formulation of claim 1, wherein the injectable liquid formulation has a pH of less than 7.

13. The injectable liquid formulation of claim 1, wherein the pH of the injectable liquid formulation in the container remains within about 5% of an initial pH measured within 24 hours of filling the injectable liquid formulation in the container as compared to a measured pH of injectable liquid formulation after storage in the container for 3 months at 40° C.

14. The injectable liquid formulation of claim 1, wherein the injectable liquid formulation comprises not more than 600 particulates having an average diameter of greater than 10 μm after storage for 3 months at 40° C.

15. The injectable liquid formulation of claim 1, wherein the injectable liquid formulation comprises not more than 0.2 weight percent of total impurities after storage for 3 months at 40° C.

16. The injectable liquid formulation of claim 1, wherein the injectable liquid formulation retains about 95% or more of the initial concentration of the lidocaine or a pharmaceutically acceptable salt thereof after storage for 3 months at 40° C.

17. The injectable liquid formulation of claim 1, wherein the injectable liquid formulation retains about 98% or more of the sodium bicarbonate buffer after storage for 3 months at 40° C.

18. The injectable liquid formulation of claim 1, wherein the injectable liquid formulation is a ready-to-use or ready-to-administer formulation.

19. The injectable liquid formulation of claim 1, wherein the container is a glass vial having a volume in the range of 10 mL to 20 mL.

20. The injectable liquid formulation of claim 1, wherein the injectable liquid formulation is free of one or more of the components selected from the group consisting of a chelating agent, a paraben, a glycol, a saccharide, a calcium salt, a sulfate, a sulfite, a sulfide, a sugar and a sugar alcohol.

21. The injectable liquid formulation of claim 1, wherein the injectable liquid formulation comprises not more than 100 particulates having an average diameter of greater than 25 μm after storage for 3 months at 40° C.

22. The injectable liquid formulation of claim 1, wherein the carrier is an aqueous carrier.

23. The injectable liquid formulation of claim 1, wherein the carrier is water present at more than 90% of the weight of the injectable liquid formulation.
